(19) United States
(12) Patent Application Publication
(10) Pub. No.: US 2015/0017715 A1
(43) Pub. Date: Jan. 15, 2015

(54) CELL COLLECTION DEVICE AND METHOD

(71) Applicant: CellTraffix, Inc., Pittsford, NY (US)

(72) Inventor: Stuart G. McDonald, Pultneyville, NY (US)

(73) Assignee: CellTraffix, Inc., Pittsford, NY (US)

(21) Appl. No.: 14/259,693

(22) Filed: Apr. 23, 2014

Related U.S. Application Data
(60) Provisional application No. 61/904,067, filed on Nov. 14, 2013.
(60) Provisional application No. 61/845,115, filed on Jul. 11, 2013.

(51) Int. Cl. C12N 5/071; C12M 1/26

(52) U.S. Cl. CPC C12N 5/0602; C12M 33/12
USPC 435/325; 435/283.1

(57) ABSTRACT

A compact and efficient cell collection device and method based on biologically-derived cell rolling and a planar collection module that provides fluid recirculation and modification of shear forces to discriminate between target and non-target cells.

### CROSS-REFERENCE TO RELATED APPLICATIONS

This application claims priority to U.S. Provisional Patent Application No. 61/845,115 (filed Jul. 11, 2013) and 61/904,067 (filed Nov. 14, 2013), the entirety of which are incorporated by reference.

### BACKGROUND OF THE INVENTION

The prior art describes numerous processes detailing the isolation of desired biological targets from bodily fluids. One such prior art reference is U.S. Pat. No. 8,105,793, hereby incorporated in its entirety. As discussed in this reference and other prior art, a biological entity of interest typically is derived from a sample that is removed from a donor, which sample contains a heterogeneous mixture of cells and other biological substances. These substances span a size scale from the macroscopic to the molecular. The heterogeneous sample is subjected to one or more separation and purification procedures in order to obtain a preparation that is enriched with the biological target. Typical heterogeneous samples from which a biological target may be derived include: peripheral whole blood, bone marrow, tumor tissue, sputum, lymphatic fluid, ascites fluid, pleural fluid, spinal fluid, urine, gastro-intestinal fluid, bile, umbilical cord blood, amniotic fluid and so forth. Often, the amount of the biological entity of interest in the sample is negligible. Therefore, the target cell, stem cells, metastatic cancer cells, viruses, prion, and so forth, must be separated and purified from an overwhelming number of very similar, often nearly identical, non-target biological entities and other unwanted biological substances. Methods for separating and purifying cells and other biological entities have been developed. So-called positive separation methods take advantage of immunoaffinity-based technology. In an immunoaffinity-based method, antibody specific for a biological entity, for example a cell-type of interest, is linked to the surface of a solid such as a particle or filtration membrane. The captured cells, that is, cells bound to the solid through bonding to the antibody, are then separated from non-bound cells by filtration, adsorption on a column, partitioning in a magnetic field, centrifugation, and so on.

Stem cells are cells capable of both indefinite proliferation and differentiation into specialized cells that serve as a continuous source for new cells for such tissues as blood, myocardium, liver, etc. Hematopoietic cells are rare, pluripotent cells, having the capacity to give rise to all lineages of blood cells. Stem cells undergo a transformation into progenitor cells, which are the precursors of several different blood cell types, including erythroblasts, myeloblasts, monocytes and macrophages. Stem cells have a wide range of potential applications, particularly in the autologous treatment of cancer patients.

Typically, stem cell products (true stem cells, progenitor cells and CD34+ cells) are harvested from bone marrow of a donor in a procedure, which may be painful, and requires hospitalization and general anesthesia. More recently, methods have been developed enabling stem cells and committed progenitor cells to be obtained from donated peripheral blood or peripheral blood collected during a surgical procedure.

Progenitor cells, whether from bone marrow or peripheral blood, can be used to enhance the healing of damaged tissues, such as myocardium damaged by myocardial infarction, as well as to enhance hematologic recovery following an immunosuppressive procedure such as chemotherapy. The use of hematopoietic and cardiac stem cells for regenerating damaged myocardium is described in PCT Publication WO2002009650. The use of human umbilical blood as a source of neural cells for transplantation is described in PCT Publication WO2001066698.

There exists a need for obtaining cellular samples from donors that are enriched in the desired biological target. Because a heterogeneous sample contains a negligible amount of a biological entity of interest, the limits of separation methods to provide viable and potent biological target in sufficient purity and amount for research, diagnostic or therapeutic use are often exceeded. Because of the low yield after separation and purification, some cell-types, such as stem cells, progenitor cells and immune cells (particularly T-cells) must be placed in long-term culture systems under conditions that enable cell viability and clinical potency to be maintained and under which cells can propagate (cell expansion). Such conditions are not always known. In order to obtain a sufficient amount of a biological target, a large amount of a sample, such as peripheral blood, must be obtained from a donor at one time, or samples must be withdrawn multiple times from a donor and then subjected to one or more lengthy, expensive, and often low-yield separation procedures to obtain a useful preparation of the biological target. Taken together, these problems place significant burdens on donors, separation methods, laboratory personnel, clinicians and patients. These burdens significantly add to the time and costs required to isolate the desired cells.

There also exists a need for obtaining cells from non-humans, which cells comprise particular antigens or antibodies of interest. The transcription and translation levels of any number of constituents, mechanical properties, in vitro memory properties or genetic properties of cells can be analyzed. Capturing immune cells, stem cells and committed progenitor cells, and metastatic cancer cells, blood borne viruses are of particular interest.

Prior art systems designed to harvest cells, particularly stem cells, from peripheral blood or treated bone marrow have in general focused on applications in research, and not on collection of large quantities of cells from larger (e.g. half-litre) sources in a timely fashion. Some of these prior art systems rely on selectin-mediated flux rolling or other cell-surface-mediated flux rolling or capture as the sole means of collection. Thus these prior art techniques are limited in the quantity of cells that may be collected by a) their internal surface area, and b) the quasi-saturation of the surface by target and non-target cells.

The discussion above is merely provided for general background information and is not intended to be used as an aid in determining the scope of the claimed subject matter.

### BRIEF DESCRIPTION OF THE INVENTION

In accordance with one embodiment of the invention, there is provided a process for collecting biological targets from the fluid of an organism comprising the steps of (1) feeding the fluid into a chamber which is coated with a target specific binding agent, (2) maintaining the flow dynamics of the primary fluid stream in order to generate flux rolling of target cells, 3) directing a minor amount of the fluid stream into a secondary fluid stream, 4) modifying the flow dynamics of the secondary fluid stream to terminate flux rolling of target cells, (5) collecting target cells from the secondary flow stream while (6) recirculating the primary fluid stream to permit multiple collection cycles of target cells.

This brief description of the invention is intended only to provide an overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic showing a fluid containing cells flowing through a chamber having cell-specific coatings on one or more walls;

FIG. 2 shows the relationship between flux rolling on a surface coated with selectin and the surface shear forces that result from changes in fluid flow rate;

FIG. 3A and FIG. 3B depicts one prior art embodiment of a cell collection device based on a geometry similar to that found in capillary vessels in living tissue, and another that is based on use of microscopic fibers;

FIG. 4 depicts a cell collection device made up of bonded sheet material and having a collection zone comprising fibrous or other material having a high surface-to-volume ratio;

FIG. 5 depicts an alternative two-layer collection device that provides convenient pumping capability;

FIGS. 6A to 6D show two pumping schemes that make use of the collection device of FIG. 5;

FIGS. 7A to 7C depict a cell collection device having multiple sheet layers and capable of self-actuation by application of pressurized fluid;

FIG. 8 shows an alternative self-actuating cell collection device having fluid pumping zones stacked together rather than side-by-side;

FIG. 9A and FIG. 9B depict a planar cell collection device and system that is based on variable control of surface shear forces;

FIG. 10 is a schematic of an embodiment the overall cell collection system of this invention;

FIG. 11 is a detailed schematic of the embodiment of the collection module component of this invention;

FIG. 12 is an assembly drawing for a prototype planar cell collection device of the embodiment. FIGS. 12 through 16 are from CAD files used in manufacturing; herein they are not dimensioned and are included for reference purposes since previous figures are not to scale;

FIG. 13 is a component drawing for the entry-side base part of the prototype collection device;

FIG. 14 is a component drawing for the exit-side base part of the prototype collection device;

FIG. 15 is a component drawing for the cover part of the prototype collection device;

FIG. 16 is an exploded view of the prototype collection device;

FIG. 17 is a schematic cross-section of two sets of components used in a stacked collection device;

FIG. 18 depicts a 20-cavity stacked collection assembly having parallel operation;

FIG. 19 and FIG. 20 depict the channel element and spacer element that make up the stacked collection assembly shown in FIG. 18;

FIG. 21 and FIG. 22 show details of the port and plug components, respectively;

FIG. 23 depicts one approach to providing for ultrasonic sealing of the stacked collection assembly;

FIG. 24 depicts a 20-cavity stacked collection assembly having serial operation;

FIG. 25A and FIG. 25B demonstrate the differences in fluid flow between parallel and serial stacked collection assemblies; and

FIG. 26 and FIG. 27 depict the channel element and spacer element that make up the stacked collection assembly shown in FIG. 24.

### DETAILED DESCRIPTION OF THE INVENTION

The term “biological target” refers to any endogenous infected or uninfected cell, or similar biological particles, including metastatic cancer cells and HIV infected cells, or virus, or bacterium, or prion, or other biological entity of interest that may be present in a fluid of a human or non-human donor or patient. Endogenous cells include but are not limited to, subsets of cells within a defined cell family, for example a B-lymphocyte or a T-lymphocyte in the lymphocyte family, or a cytolytic T-lymphocyte in the T-lymphocyte family, or an entire family of cells, such as the lymphocyte family Examples of other endogenous cells are fibroblasts, neuroblasts, hematopoietic stem cells, hematopoietic progenitor cells (CD34+ cells), mesenchymal stem cells, dendritic cells, cytolytic T-cells (CD8+ cells), other leukocyte populations, pluripotent stem cells, multi-potent stem cells, embryonic cells or islet cells. Biological targets include populations of cells having distinct phenotypic characteristics: B-cells, T-cells, NK cells, other blood cells, neuronal cells, glandular (endocrine) cells, bone forming cells (osteoclasts, etc.), germ cells (e.g., oocytes), epithelial cells lining reproductive organs, trophoblastic and placental cells in amniotic fluid and mesenchymal progenitor, neuronal progenitor, neuroectodermal cells. A biological target such as a leukocyte, stem cell or an insoluble protein may be in suspension within a fluid of a donor or it may be dispersed as a microscopic colloid, such as a large soluble protein or it may be in true molecular solution, such as a small molecule.

The term “target specific binding agent” refers to a molecule or fragment of a molecule that binds to a particular biological target. A target specific binding agent may bind a cell surface moiety, such as a receptor, an antigenic determinant, an integrin, a cell adhesion molecule, or other moiety present on a cell-type of interest. A binding agent may be specific for a region of a protein, such as a prion, a capsid protein of a virus or some other viral protein, and so on. A target specific binding agent may be a protein, peptide, antibody, antibody fragment, a fusion protein, synthetic molecule, an organic molecule (e.g., a small molecule), or the like. In general, a target specific binding agent and its biological target refer to a ligand/anti-ligand pair. Accordingly, these molecules should be viewed as a complementary/anti-complementary set of molecules that demonstrate specific binding, generally of relatively high affinity. Cell surface moiety-ligand pairs include, but are not limited to, T-cell antigen receptor (TCR) and anti-CD3 mono or polyclonal antibody, TCR and major histocompatibility complex (MHC)+antigen, TCR and super antigens (for example, staphylococcal enterotoxin B (SEB), toxic shock syndrome toxin (TSST), etc.), B-cell antigen receptor (BCR) and anti-immunoglobulin, BCR and LPS, BCR and specific antigens (univalent or polyvalent), NK receptor and anti-NK receptor antibodies, FAS (CD95) receptor and FAS ligand, FAS receptor and anti-FAS antibodies, CD54 and anti-CD54 antibodies, CD2 and anti-CD2 antibodies, CD2 and LFA-3 (lymphocyte function related antigen-3), cytokine receptors and their respective cytokines, cytokine receptors and anti-cytokine receptor antibodies, TNF-R (tumor necrosis factor-receptor) family members and antibodies directed against them, TNF-R family members and their respective ligands, adhesion/homing receptors and their ligands, adhesion/homing receptors and antibodies against them, oocyte or fertilized oocyte receptors and their ligands, oocyte or fertilized oocyte receptors and antibodies against them, receptors on the endometrial lining of uterus and their ligands, hormone receptors and their respective hormone, hormone receptors and antibodies directed against them, and others.

The term “enriched” means that the amount of biological target contained in a unit volume of donor fluid from which it is derived is less than the amount contained after release from an implanted target specific capture device or multiple devices and reconstitution into an identical volume of suitable liquid medium. The term “sufficiently enriched” means there is a sufficient amount of biological target within an implanted device or multiple devices such that the biological target may be used directly in a research, diagnostic or therapeutic application for which it is intended, or there is a sufficient amount of biological target so as to significantly reduce the amount of sample required to obtain a useful preparation using conventional separation and purification methods such as those noted earlier.

The term “fluid communication” means that two objects A and B are related such that a pathway, conduit, channel or passageway exists between objects A and B enabling a volume element of fluid at a locus of object A to flow, move, pass, be conducted or transported, along the pathway, conduit, channel or passageway to a locus b of object B. The pathway, conduit, channel or passageway may be linear, non-linear, convoluted, or be of any form as long as a volume element of fluid can pass from object A to object B.

The term “chemical attractant” or “chemoattractant” means a substance capable of luring a biological target that is capable of migration to the capture zone. One or more chemical attractants may be included in a target specific capture device. Reference may be had, e.g., U.S. Pat. No. 6,419,917; U.S. Pat. No. 6,274,342; U.S. Pat. No. 6,458,349; U.S. Pat. No. 6,320,023; and U.S. Pat. No. 6,207,144.

The term “margination” refers to the migration of a biological target from a position in its carrier fluid to the wall of the channel that carries the fluid. The concentration of marginated biological targets will be enriched near the walls of the channel. An agent is deemed to be margined when its translational velocity is below the critical hydrodynamic velocity (V_{crit}) at a radial position one cell radius from the channel wall. The critical hydrodynamic velocity for a given tube may be calculated from the Navier-Stokes equation:

$${V}_{crit} = ( \frac{2 Q}{{D}^{2}} ) ɛ ( 2 - ɛ )$$

$$where$$

$$ɛ = \frac{{D}_{cell}}{{D}_{channel}}$$

One means for measuring the translation velocity of an agent is taught elsewhere in this specification.

The term “flow dynamics” refers to the wall shear rate, the flow rate, and the translational velocity of a specified particle within a moving fluid. To modify the flow dynamics, at least one of the aforementioned properties must be altered.

A method and device are provided for sequestering a biological target from a donor, the method comprises utilizing a target specific device, wherein the target specific device comprises a region comprising immobilized biological target specific binding agent or chemical attractant. Internal fluid of the donor, which fluid comprises a biological target, contacts the device and biological target in the fluid interacts with the immobilized biological target specific recognition agent. The acts of recognition and interaction alter the movement of the biological target in question. In one embodiment, the flow characteristic so varied is modified in different ways in separate sections of the device so as to maintain rolling contact between the biological target and the target specific binding agent in one section of the device, and to eliminate rolling contact in another section of the device. One may so vary flow characteristics such as, e.g., the fluid velocity, the distribution of various components within the bodily fluid, and the shear stress of the fluid. The method and device of this invention are designed to permit the primary amount of donor fluid to recirculate through the device in order to permit multiple opportunities to engage biological targets in rolling contact and eventual collection, wherein the collected biological targets exit the device in a relatively small fraction of the initial fluid volume, thus significantly enhancing the collection efficiency of the device and permitting it to be small in physical size and cost.

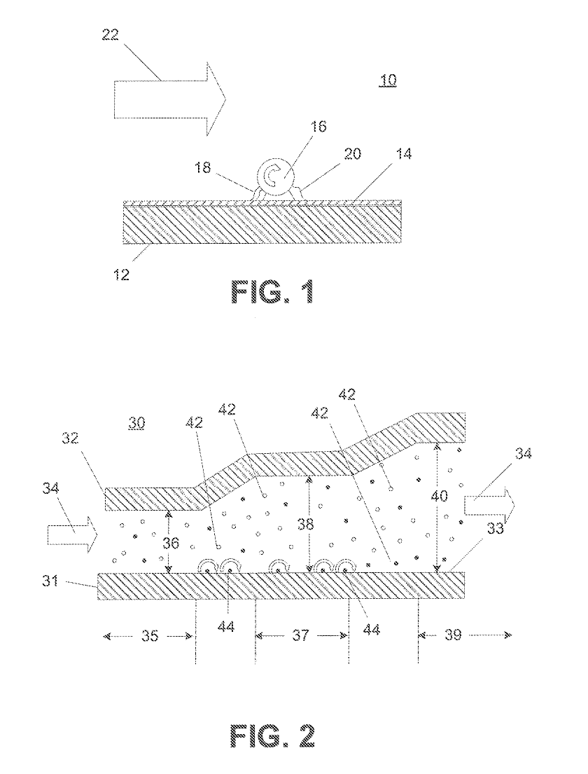

FIG. 1 is a schematic representation of the behavior of a cell in rolling contact with a surface, said rolling contact being mediated by cell-specific molecules that are adsorbed or by other means fixed to a substrate surface. As previously referenced and disclosed there are molecules such as selectins and other naturally occurring cell-surface-specific compounds that create an attractive force between a cell and a surface that results in flux rolling rather than static bonding. Referring to FIG. 1, the local region 10 shows a substrate 12 that may be metal, glass, polymer, or other material upon which is coated a thin layer 14 comprising selectin or other previously disclosed adherent molecules. Vector 22 depicts flow of the fluid surrounding cell 16, said flow resulting in shear forces that tend to move the cell in the direction of flow, and also to remove it from the thin layer 14. The biochemical reaction between cell 16 and thin layer 14 results in transitory leading bonds 20 at the leading side of the cell during rolling, and transitory trailing bonds 18 at the trailing side of the cell during rolling. In practice, if the flow rate creates kinetic energy resulting in a surface shear force significantly larger than 2.5 dyne/cm² the bonds between cell 16 and thin layer 14 are easily broken and the cell migrates away from thin layer 14. If the flow rate creates kinetic energy resulting in a surface shear force reasonably close to 2.5 dyne/cm², the transitory trailing bonds 18 and leading bonds 20 will be sufficient to cause cell 16 to remain in contact with thin layer 14, but the kinetic energy will be sufficient to cause trailing bonds 18 to gradually break while new leading bonds form; thus the cell will migrate slowly along the surface of layer 14 in the direction of flow indicated by vector 22.

If however, the flow rate is reduced to a level significantly below the 2.5 dyne/cm², to a level approaching or below 0.25 dyne/cm², the biochemical interaction between the surface of cell 16 and thin layer 14 never results in formation of trailing bonds 18 and leading bonds 20; the result is that either the cell never engages in flux rolling, or if it has done so, flux rolling terminates and cell 16 moves back into the bulk fluid.

It should be noted that the interaction between cell 16 and selectin-coated thin layer 14 as shown schematically in FIG. 1 is specific to a small population of target cells such as stem cells or circulating cancer cells, as discussed earlier. Other non-target cells that often comprise the vast majority of cells in bulk flow will never engage in flux rolling no matter what the flow kinetics are.

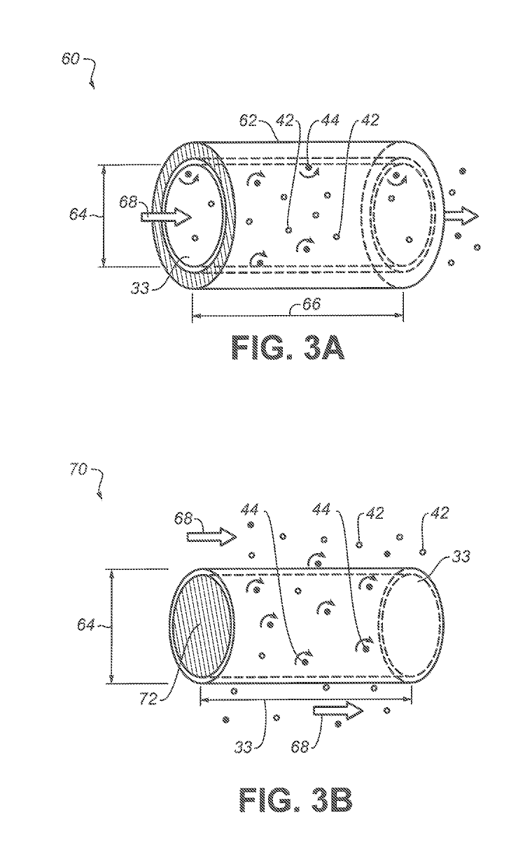

FIG. 2 is a schematic description of a practical implementation of the flux rolling process described above, showing how a microscopic channel may be designed to create and then eliminate flux rolling. Although in a real application the vast majority of cells will not be target cells, for purposes of the current discussion it is assumed that all cells 42 and 44 shown in FIG. 2 are target cells (e.g. stem cells). Channel 30, having a lower wall 31 and an upper wall 32, contains fluid flow indicated by flow vector 34. Lower wall 31 is coated with an attractant chemical 33 (e.g. selectin) material per previous discussion. For a fixed flow rate, surface shear force will be an inverse function of channel height. As shown in FIG. 2, channel height 36 in zone 35 results in surface shear force significantly higher than 2.5 dyne/cm², and it can be seen that none of the target cells 42 engage in flux rolling but rather remain in bulk flow. Channel height 38 in zone 37 of channel 30 is chosen to result in surface shear force that is approximately 2.5 dyne/cm², and it can be seen that some portion of target cells 44 engage in flux rolling, while others not in the immediate boundary layer above surface do not have the opportunity to engage in flux rolling. Also if a large number of target cells engage in flux rolling they may partially saturate the surface, denying other target cells the opportunity to engage in flux rolling. As would be apparent to those skilled in the art after benefiting from reading this specification, if channel 30 is sufficiently long, virtually all target cells may be eventually attracted to attractant chemical 33. However, this may be prohibitively expensive and create difficulties in eventual harvesting of target cells, so other means to reduce size and cost are discussed elsewhere in this specification.

Returning to FIG. 2, if channel height 40 in zone 39 of channel 30 is selected to create a shear force at the attractant chemical 33 that is significantly below 2.5 dyne/cm² it can be seen that all target cells 42, including those that were previously engaged in flux rolling in zone 37, separate from attractant chemical 33 and migrate away in bulk flow.

In some embodiments of this invention, target cells are engaged in flux rolling, then for purposes of harvesting them secondary chemical compounds are utilized to strip them from the attractant surface. In certain embodiments of this invention, secondary compounds are not required; rather the consumables used in collecting target cells are specifically designed to manipulate surface shear forces to harvest cells. As will be seen later in this disclosure this permits recirculation of cell-containing fluid and multiple re-use of the attractant surface, reducing size and cost of the consumable significantly as well as increasing the enrichment of target cells that are collected.

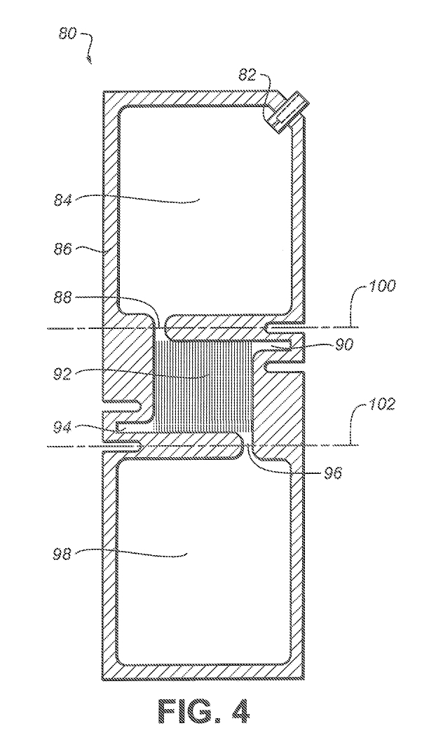

FIG. 3A and FIG. 3B depict two options for flux rolling and eventual harvesting of target cells. FIG. 3A depicts a section 60 of a miniature or microscopic tube 62 having an internal diameter 64 that may be as small as 50 um or as large as 1 mm but preferably in the range of 100 to 300 urn, and more preferably around 200 um, as has been shown in experiments described in references cited herein. Tube 62 has an interior attractant chemical 33 of selectin or other attractant chemical, and has a fluid flow velocity 68 such that in combination with the internal diameter 64 the resulting surface shear force is approximately 2.5 dyne/cm². Overall length 66 of tube 62 will create a surface area; either a single long tube may be used (largely for research purposes) or a large population of tubes 62 may be affixed in parallel to increase the surface area so that a sufficient number of target cells 44 may engage in flux rolling and eventually be harvested.

FIG. 3B depicts a section 70 of a fiber 72 that is made up of glass, metal, or a polymer, that has an outer diameter 64 that may be the same as the inner diameter of tube 62 in FIG. 3A, and that has the same attractant chemical 33 on its external surface as tube 62 of FIG. 3A does on its internal surface. It may be seen that cells 42 in bulk flow may be attracted to attractant chemical 33 and engage in flux rolling on the surface in similar manner as within tube 62 providing that the shear force on the exterior of fiber 72 is in the range of 2.5 dyne/cm². In the case shown in FIG. 3B shear force will be a function of fluid flow rate as shown by vector 68 but also will be a function of the proximity of fiber 72 to other identical or similar fibers (not shown). As will be apparent to those skilled in the art, multiple fibers 72 may be arranged in a linear fashion (as in dialysis or cell culture modules) or may be in the form of a matt. While flow will vary depending on orientation and spacing of fibers, it will be apparent that some portion of target cells will engage in flux rolling and may eventually be harvested using secondary chemicals to strip them from the surface of fibers 72 in similar fashion to chemicals used to strip cells from the interior of tubes 62.

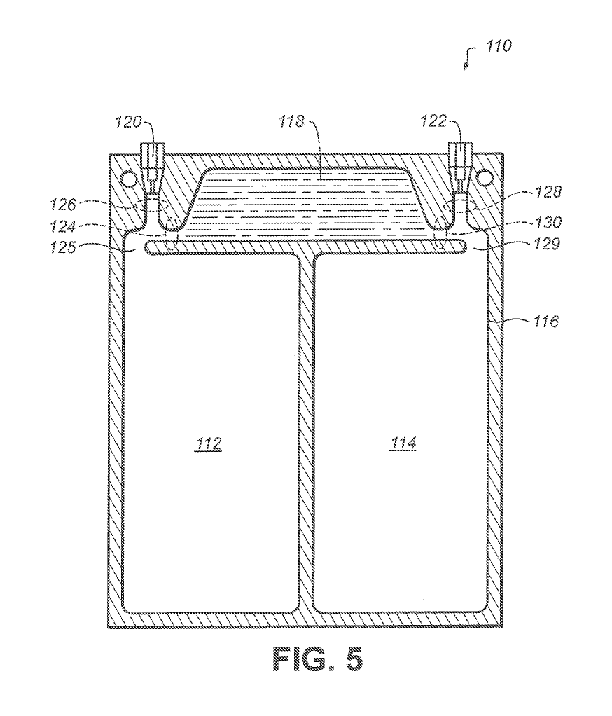

FIG. 4 is a conceptual drawing of one embodiment of this invention, and it should be noted it is not drawn to scale with respect to the practical need to process a sample on the order of one pint (or 0.5 litre) of peripheral blood or prepared bone marrow. The collection module 80 is a three-zone laminar bag similar to those used to supply blood products and other fluids. Sealing may be ultrasonic, simple thermal, adhesive, or a combination of these. The use of three zones in based on the expectation that per-pass collection efficiency of the selectin binding technology, even with halloysite and/or other surface modification in the collection zone 92, will be relatively low; on the order of 15%. Thus multiple passes through the collection zone can raise the eventual collection efficiency to near-100%. Input port 82 is sealed into the laminar bag structure, and may be in the form of a standard luer fitting, and preferably will also have a duckbill or other form of check valve in order to prevent backflow during operation. The collected sample (either peripheral blood or pre-treated bone marrow) is injected into this port. Seal area 86 is shown in this figure as being a fairly regular and linear. However in order to permit efficient and complete pumping, to properly seal around specific features such as input port 82, and to permit pinch-valving, seal area 86 will likely have a more complex shape than is shown in this figure. Pumping zone 84 is designed to be large enough to hold the entire volume of fluid being processed. In order to generate the appropriate pressure to create the desired shear forces for collection, pumping zone 84 may be simply exposed to force applied by a platen that squeezes its surfaces together. This force may be applied via a mechanical drive (e.g. lead screw, cam, hydraulic, pneumatic) or by a passive drive (e.g. weight, spring) that can be manually or mechanically activated repeatedly. Pumping zone 84 fluidly communicates with collection zone 92 through channel 88. This channel is placed near a diagonal corner of these zones in order to facilitate complete emptying and efficient flow. This approach is also taken in other areas of the consumable. Channel 88 may also be used as a valve position; after collection is completed the cells collected in collection zone 92 will be exposed to an elution fluid flowing transverse to the flow direction used during collection; during the elution process the pumping zones 84 and 98 will need to be closed off. This closure may be accomplished by a simple pinch valve pressing channels 88 and 96 closed, and alternatively may be accomplished permanently by a thermal or ultrasound seal (with or without adhesive applied to the surfaces of the channel being sealed). Collection zone 92 may comprise a large number of hollow tubes or a similar number of fibers. Fibers may be arranged in a linear fashion similar to filter tow or as a matt similar to depth filters used in industry. If microtubes are used, additional design features will be required to force the fluid to flow internal to, rather than external to, the tubes. Fibers (in either filter tow or matt format) or microtubes may be pre-assembled into a cartridge (not shown) or simply assembled into the laminar package at the time of bonding the top and bottom surfaces.

Recognizing that clogging and other causes of non-uniform flow may degrade collection efficiency, the design of collection zone 92 must reflect the potentially conflicting demands of high surface area (as much as 0.5 m² for a single-pass operation), uniform shear (near 2.5 dyne/cm²), effective cross-flow elution, uniform collection of cells over the entire volume, and robust handling. As fluid flows from pumping zone 84 through collection zone 92, it transits channel 96 (also a site for pinch valve or seal) into second pumping zone 98. Pumping zone 98 has a volume similar to pumping zone 84. The approach to actuating pumping zone 98 may be identical to that used to actuate pumping zone 84, however, in order to reduce design complexity a passive approach may be preferred. If pumping zone 98 has a steady-state force applied that is consistent with a desired shear level in collection zone 92 during return flow into pumping zone 84, then pumping zone 84 may be actuated by alternating between force levels of zero and twice the desired shear level. Thus only one set of active actuating hardware and controls is needed (e.g. over pumping zone 84) while a simpler passive approach may be taken over the other zone (e.g. pumping zone 98).

If there are no biochemical, kinetic, clogging, or saturation limitations to subsequent per-pass efficiency, cyclic actuation of the system may be used to increase efficiency to near-100%. By example, if each transit has a collection efficiency of 15%, four cycles (8 transits through collection zone 92) could increase collection to over 70% and eight cycles could increase it to over 90%. Once collection cycling is complete, collection zone 92 may be compressed to remove any remaining fluid while retaining all collected cells. Then channels 88 and 96 are closed as discussed above by pinch valving or permanent sealing. Cell elution ports 90 and 94 are shown in FIG. 4 as simply areas of the laminar consumable where access may be had simply by cutting across the exposed tab. Alternatively, luer fittings (with or without check valve) may be used to permit fluid access and removal. In the simplest case, the elution fluid may be introduced through a luer/check valve in elution port 90 thus re-inflating collection zone 92 with elution fluid. After an appropriate holding time, the fluid containing the collected stem cells may be removed through elution port 94. In order to completely harvest the collected cells, more than one elution cycle may be used or a continuous flow approach from elution port 90 to elution port 94 may be employed. In addition, application of vibrational or thermal energy may increase the efficiency of cell recovery. In order to reduce the size of the pumping hardware (not shown) and potentially simplify its design, one may consider ‘folding’ the consumable in two places; one between pumping zone 84 and collection zone 92, and also between collection zone 92 and pumping zone 98 (shown by dashed lines 100 and 102 respectively). Two folds of 90° at these locations will place pumping zones 84 and 98 adjacent and parallel; the actuation means may be placed between them and may act against rigid walls outboard of the two zones. In this case, care must be taken not to pinch off flow through channels 88 and 96 during collection cycling while also permitting temporary or permanent sealing of these channels during the cell removal process.

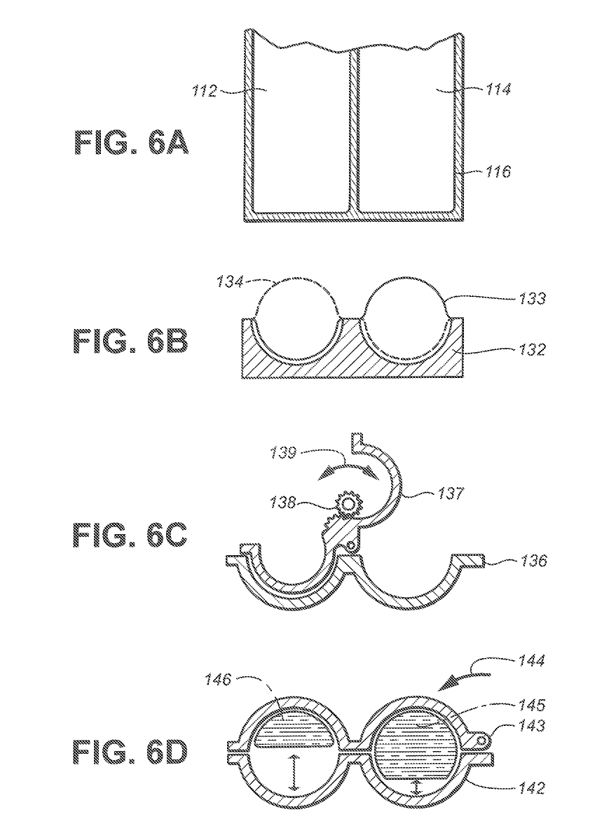

FIG. 5 depicts an embodiment similar to that in FIG. 4 but with a different geometry and is sized so that pumping zones 112 and 114 will hold approximately 0.5 litres of fluid when filled. Consumable 110 is made of a laminated flexible material, preferably a polymer, and is sealed using ultrasonic welding, adhesives, or a combination of these, as shown by seal area 116 in FIG. 5. Pumping zones 112 and 114 communicate through collection zone 118 through channels each having two pinch valves; valves 124 and 125 between zones 112 and 118, and valves 129 and 130 between zones 118 and 114. It may readily be seen that initial injection of sample material through input port 120 into zone 112 may be effected with valves 125 and 126 open and valve 124 closed. Cycling of sample material between zones 112 and 114 for collection purposes may be done with valves 126 and 128 closed and valves 124, 125, 129, and 130 open. After all collection cycles between zone 112 and 114 are completed, elution fluid may be injected into collection zone 118 via input port 120 and removed via exit port 122 with valves 124, 126, 128, and 130 open and valves 125 and 129 closed.

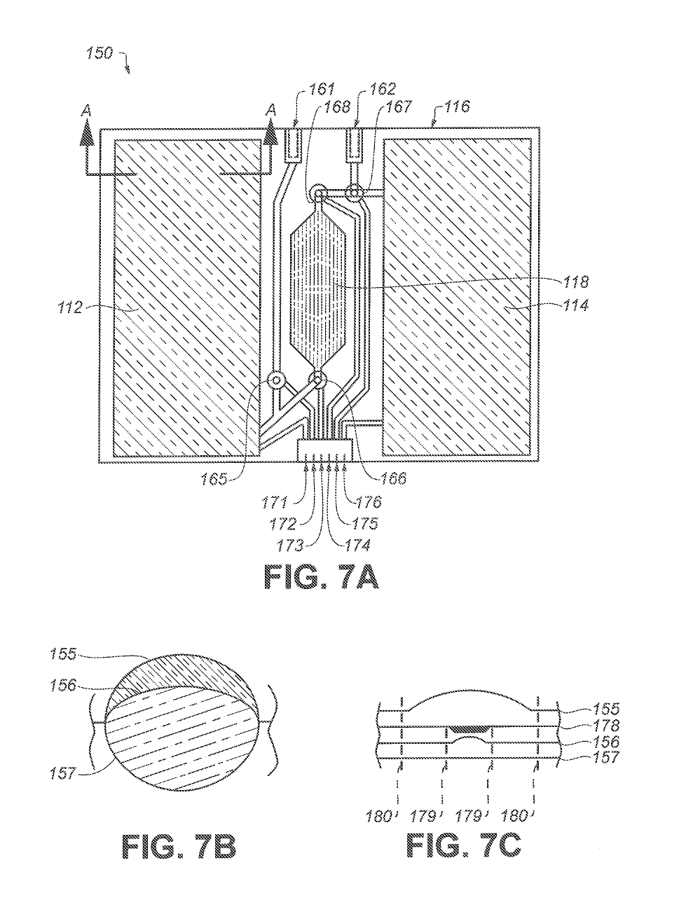

FIGS. 6A to 6D show two of a number of different options for operating the collection device shown in FIG. 5. FIG. 6A is a partial plan view of the three-zone laminar bag used as a stem cell collection consumable. Pumping zones 112 and 114 are shown in approximate alignment with like zones in the FIG. 5. Seal area 116 is shown as in FIG. 5 but it should be noted that for the proposed pumping action in the two embodiments described here to function the seal area separating pumping zones 112 and 114 may be wider than shown due to relative motion as the laminates structure is compressed. It may also be helpful to have two or more alignment holes in the central seal area to provide alignment of the consumable to the drive unit (not shown). This approach may be applied to either drive concept discussed here, or any other not shown. FIG. 6B is a section view of the consumable held in a nest 132. Solid outline 133 depicts the consumable with the right hand side full and the dashed outline 134 depicts the left hand side full. Pumping through the cell collection zone (not shown in these FIGS. 6A-6D) will be achieved by applying force to left and right portions of the consumable in a cyclic fashion; each cycle achieving two transits of all fluid through the collection zone. As discussed elsewhere, in order to create flow through the collection zone having well controlled shear at or around 2.5 dyne/cm² a relatively uniform hydrostatic pressure applied to the collection module during cycling will create optimal cell collection. Setpoint control and potential for adjustment will be helpful during product development.

FIG. 6C depicts one embodiment of a drive that applies mechanical force in a manner that creates controlled pressure in the consumable zone being compressed. This figure depicts a rocker approach but other (e.g. linear force applied to a platen or a peristaltic roller; both not shown) may also be used. In this embodiment the vacuum-formed consumable bag having a naturally curved shape is supported in nest 136 and rocker 137 is driven by gear 138, its cyclic motion indicated by arrow 139. A four-bar link, cam drive, or belt drive may also be employed to generate the cyclic rotation of rocker 137. In order for uniform hydrostatic pressure to be generated within the chamber being compressed, some form of mechanical force feedback may be required if a direct mechanical drive is employed.

FIG. 6D depicts another embodiment that provides an easy way to control hydrostatic pressure. Base 142 supports the consumable and cover 143 closes over it as shown by arrow 144. Bladder 145 is shown almost completely full and bladder 146 as being only partially full. At any time the combined volume of bladders 145 and 146 should approximate the full volume of one side of the base 142 and cover 143 assembly. A primary advantage of this approach is that the pressure generated within the consumable chamber being driven will be almost exactly the same as the pressure in the bladder being used to compress it, the difference being elastic and hysteresis forces in the wall of the consumable itself. These offsetting forces can be held to near zero with proper choices of consumable wall material, shape, and assembly technique. One approach would be to activate bladders 145 and 146 pneumatically, with active pressure control. In this case one alternative would be to have an air tank (not shown) that accumulates gas at a higher pressure than required to actuate the consumable, and pressure feedback used to control valves between the tank and bladder. The advantage here is that the pneumatic pump will not need to operate full time, and one alternative is to pre-charge the tank with sufficient volume and pressure of gas that the full number of cycles (e.g. 8 cycles and 16 transits of fluid through the collection zone) could be achieved without any gas pumping action. This alternative will be attractive in a remote setting or a bedside setting, where all other system functions may easily be powered by a small battery. A second approach to activating bladders 145 and 146 would be hydraulic. This will be more energy efficient than a pneumatic approach since valve losses will not exist. It may also be easier to control hydrostatic pressure with a hydraulic approach, and even though pumping will be constant in this case, the energy levels may be readily achieved in a remote setting with a reasonably sized battery pack.

In both of the embodiments shown in FIGS. 6C and 6D thermal control, pinch (or other) valving, and porting of sample and elution fluids are not shown since they are straightforward.

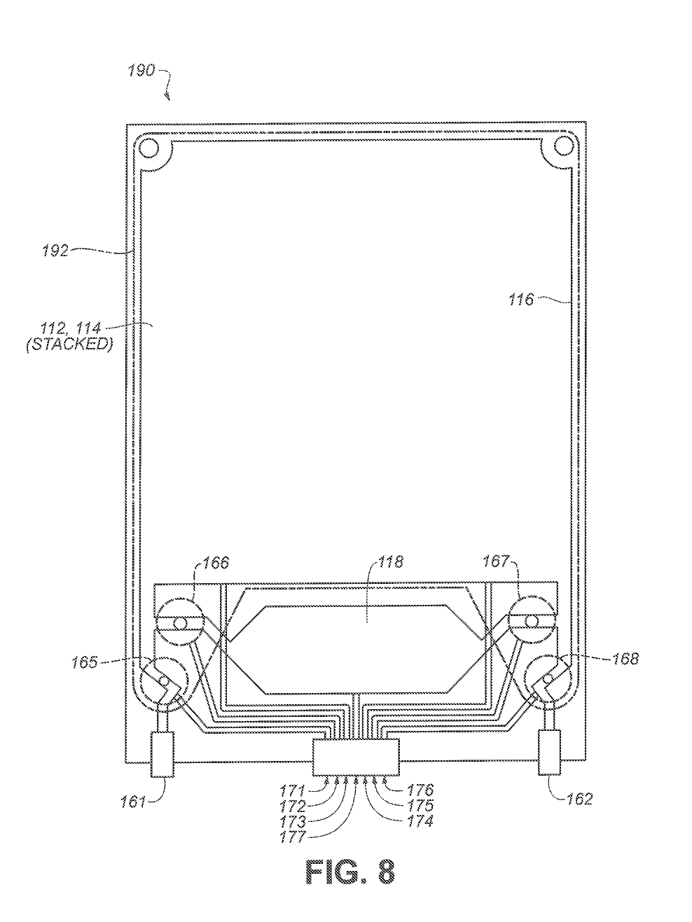

FIG. 7A is a plan view of a cell collection device similar in operation to that shown in FIGS. 4 and 5 except that a drive unit that actually houses the consumable is not required. The embodiments shown in FIGS. 4 and 5 would also use a dedicated drive unit for each unit of blood or other fluid being processed, or a larger drive unit having a sufficient number of modules or nests to process multiple units of fluid at the same time. Given that thermal control requirements may be simple (likely room temperature) and that as shown in FIG. 6D hydraulic or pneumatic energy can be used to cause pumping action, a relatively straightforward application of pneumatics may be used also to actuate the required pinch valves. Extrapolating from this point, the embodiment shown in FIG. 7 eliminates all housing requirements; the topology provides for two fluid ports, input port 161 and exit port 162, in addition to six pneumatic ports that can be arranged in a single connector. Given also that the variability in blood viscosity is minimal, multiple consumables 150 as shown in FIG. 7 may readily be processed using a fixed cycle time. This in turn creates the opportunity for a single pneumatic drive unit having sufficient volumetric output to address multiple six-port pneumatic connectors to process multiple target cell collection modules simultaneously. In order to lay out the consumable so that pneumatic ports may be integrated into a single connector, FIG. 7 shows the two pumping zones 112 and 114 separated laterally and the collection zone 118 between them. Otherwise the operation of consumable 150 in cycling to collect stem cells is essentially identical to consumable 110 shown in FIG. 5. The reader is directed to the discussion of FIG. 5 above for operational details; this discussion will focus on features found in cell collection consumable 150 that obviate the housing requirements of the driver and simplify it to the point of being a timed pneumatic source.

Operational simplification is achieved by the addition of a third laminate layer to consumable 150. This configuration provides two fluid-containing areas. In one embodiment, the fluids are both liquids. In another embodiment, one fluid is a liquid while the other is a gas. In the section view shown in FIG. 7B layers 157 and 156 are the same as in consumable 110 and the volume between layers 156 and 155 is the volume that creates the steady pressure that causes blood flow through the collection zone 118. As the space between layers 156 and 155 is pressurized, blood held between layers 156 and 157 is forced through the collection zone 118 and fills the other pumping zone 114. Port 171 is used to supply air to pressurize the left pumping zone 112; when pumping is complete for the current cycle, port 171 is opened to atmospheric pressure and port 176 is then pressurized to reverse flow from pumping zone 114 to pumping zone 112. This cyclic process is repeated for the number of cycles (e.g. 8) required to effect complete harvest of stem cells in the sample. Optionally, the current blood volume may be disposed of and another placed in the consumable for collection of additional cells, and as an alternative, pretreated bone marrow may be used in place of peripheral blood.

Since the consumable 150 is not held in a nest, valving may be accomplished without external mechanical support. This is shown in the FIG. 7C as a magnified cross-section of one of the four valves 165, 166, 167 and 168 that are integrated into the consumable. The detail is shown as an exploded view of four layers that are bonded using ultrasound, adhesive, or a combination of both. Layers 155, 156, and 157 are the same as in all other regions of consumable 150. Layer 178 is an additional patch that is placed between layers 155 and 156 prior to sealing. The slight curved central region in layer 156 as shown in FIG. 7C is actually a cross section of the channel being pinched by the valve. Linear intra-layer bonding positions 179 form the channel between layers 156 and 157 that is being controlled by the pinch valve. A circular intra-layer bond between layers 155 and 178 is shown in FIG. 7C at its circumference 180 forms the larger domed volume that may be pressurized to actuate the pinch valve. It may be seen in FIG. 7A that each valve 165, 166, 167, and 168 is connected via a small air channel with matching ports 172, 173, 175, and 174 respectively. Since the circular actuating air volume created by circumference 180 is four times larger in diameter than the section 179 of the channel being pinched, the pinching force applied by the button shown attached to layer 178 is created by an area 4² or 16 times greater than the area of the channel being pinched. Thus there is sufficient force to close the channel while using relatively low air pressure. Optionally, a small rigid disc (not shown) may be bonded to the bottom of layer 157 so that layer cannot deform in a way that defeats closure of the channel.

Valves 165 to 168 are used in similar manner to the valves shown in FIG. 5 and are connected via small channels created between layers 155 and 156 to ports 172 to 175. Air pressure used to pump fluids is carried through similar channels formed between layers 155 and 156 from ports 171 and 179 in order to provide pumping pressure to pumping zones 112 and 114 respectively. Thus a single 6-port connector having a shape that prevents reverse attachment is sufficient for connecting to a drive unit. As discussed previously, actuation of valves may be done on a fixed timing basis as long as sufficient time is allowed for pumping a relatively high-viscosity unit of sample fluid; lower viscosity units will be pumped more quickly so there is no loss in overall system efficiency due to timing issues. The volume of air required to pump blood will be at least two orders of magnitude higher than that require for valve actuation, and given the design of the valves themselves the same pressure may be used. So processing of ‘N’ samples may be carried out with a drive unit having one pump, one set of control logic and valves, as long as the volumetric output of the drive unit pump is ‘N’ times the output required to pump a single collection consumable.

If multiple units of blood are processed in parallel, but it is desirable that not all are started at the same time, the control algorithm will need to provide for a dwell period needed for introducing new unit(s) into the processing cycle. In addition the algorithm (not discussed herein) must provide control during the cell elution process that is different from the nominal pumping process.

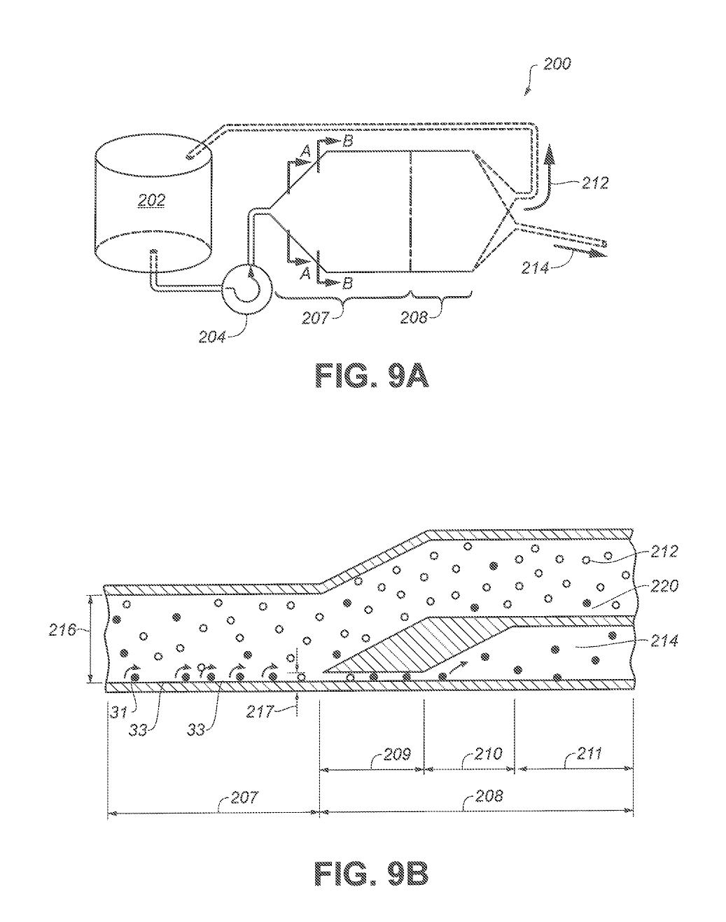

FIG. 8 depicts a cell collection consumable 190 similar in operation to previously disclosed consumables 110 and 150 shown in FIGS. 5 and 7. The embodiment shown in FIG. 8 provides a more compact form factor, improved valve operation, provision for applying pressure to the collection zone, and provision for pulsatile flow. Cell collection consumable 190 has reduced area since pumping zones 112 and 114 are stacked rather than side by side. This requires a rigid support so that upon bladder inflation to achieve pumping action of one chamber, the other chamber must not also be compressed. This is achieved by a multi-layer laminate assembly having in one embodiment seven layers in the consumable, and in the other embodiment an external platen providing central rigidity. Consumable 190 as shown in FIG. 8 has pumping zones 112 and 114 having the same outline, and dashed line 192 indicates the exterior shape of a rigid central platen that may be part of the consumable, or may be an external support plate that slips into the consumable from one side, providing a rigid central plane and also providing physical support for the consumable during actuation. On the front side of the platen (toward the reader) there are two layers that contain the blood volume, and a third layer that is filled with air to provide pumping action. This arrangement is repeated on the reverse side of the platen. If the two air chambers are inflated alternately, blood will be pumped in a cyclic fashion through the collection zone as in operation of consumables 110 and 150 described previously. This approach requires slightly larger area for the pumping zones since they will inflate more like a pillow than a cylinder due to the presence of the platen. However, since they are stacked the overall form factor of this consumable is significantly smaller than previous concepts; this is both a practical and a human factors improvement.

The dashed outline of the platen 192 extends down on both sides of consumable 190 to provide backing for the four valves 165 to 168, guaranteeing effective valve operation without any external support or backing. This approach may make lateral insertion of an external support platen topologically difficult; in this event small separate rigid discs may be integrated into the design to achieve the same improvement in valve operation. It will be noted that in consumable 190 there are seven rather than six pneumatic ports in the connector at the bottom of the consumable. Ports 171 to 176 in consumable 190 are used to carry out the same functions as ports having like numbers in consumable 150, and port 177 in consumable 190 communicates with a third laminate layer associated with the collection zone, providing the opportunity to pressurize a bladder disposed immediately lateral to that zone. Multiple operational improvements can be realized:

Alternate inflation and deflation of the bladder (not shown) over the collection zone 118 can be used to improve the efficiency of the post-collection cell elution and removal process.

If collection zone 118 is held under pressure by the bladder during normal cell collection it may be optionally depressurized to eliminate any clogging between fibers that may have occurred during multiple cell collection cycles.

Since pulsatile flow is associated with natural blood flow and cell adhesion processes, its effect in modulating kinetic energy and thus surface shear forces may provide improvement in selectivity of cell collection since the variation in shear rate will lead to a response similar to bound/free separation used in immunochemistry.

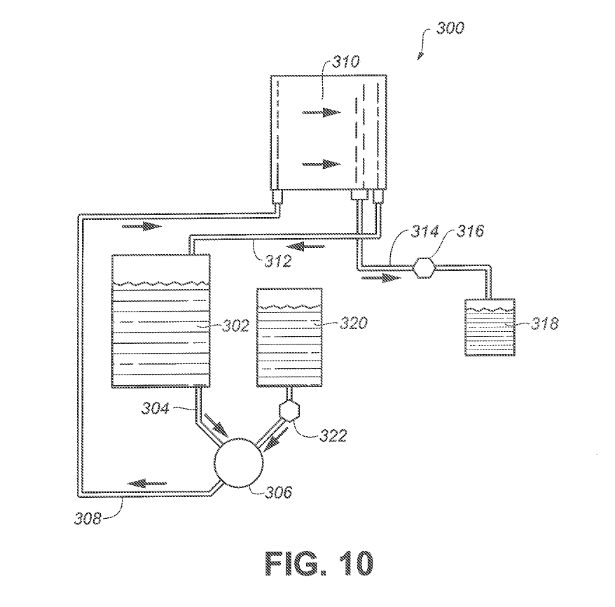

FIG. 9A is a system-level schematic of an embodiment of this invention and FIG. 9B is a cross-section view of the critical region of the collection module component of the invention. As previously discussed it has been observed that it is possible to manipulate the flux rolling behavior of target (e.g. stem) cells over a surface coated with attractant (e.g. selectin) chemistry simply by modifying the surface shear force by varying flow velocity. The key insight is that while the ‘sweet spot’ for rolling behavior of target cells over a selectin-coated surface is around 2.5 dyne/cm², rolling does not occur at significantly higher shear force levels nor at significantly lower shear force levels. The lack of rolling behavior at much higher shear force levels is simply due to local kinetic energy that defeats the transitory bonds that cause rolling on the selectin-treated surface, and the prevailing opinion has in the past been that at much lower levels of kinetic energy there would be a relatively high degree of unwanted nonspecific binding of non-target cells. This latter assumption appears to be incorrect; local shear forces below 0.4 dyne/cm² result in biochemical interactions that may permit occasional tethering behavior, but not the rolling behavior that is needed to selectively attract and collect target cells. A second key insight comes from the fact that after target cells are removed from the selectin surface, that surface may be re-used with essentially the same efficiency as found in the first pass. This parallels the reality in vivo, where cell rolling behavior in the healing process found in vertebrates permits interaction between multiple cells over the same capillary vessel surface. Thus, in a cell collection system, if cells can be somehow removed from the selectin surface, that surface may be used in steady-state mode; each site interacting with a large number of rolling target cells over an extended period of time.

Referring to FIG. 9A, cell collection system 200 comprises a source 202 of peripheral blood or bone marrow, pre-treated as needed, and pumped by pump 204 through a collection chamber 206 (shown in plan view) that is planar in general. There are two outputs of the chamber; recirculation output 212 recirculated to the source where it re-mixes and cycles back through collection chamber 206, and harvest output 214 carries harvested target cells to a container (not shown) for storage and/or therapeutic use. The ratio of flow between the recirculation output 212 to the harvest output 214 may be as high as 100:1 or more, so the ‘average’ cell or fluid volume may pass through collection chamber 206 as many as 10 or more times before the fluid volume has been significantly reduced. In FIG. 9A, two cross-sections (labeled A-A and B-B) are shown as well as a collection zone 207 and a harvest zone 208. Section views A-A and B-B are not shown but the premise is that the cross-sectional flow area in them is essentially the same as the inlet conduit area, gradually transitioning from a small round inlet to a wide and very thin planar geometry at the beginning of collection zone 207.

Referring now to FIG. 9B that is a side view, collection zone 207 may be on the order of 100 um to 1 mm in thickness (dimension 216) and is preferably about 200 urn. Its lower wall 31 is coated with selectin or other attractant chemical 33 in order to induce rolling behavior in cells that come within the nominal capture distance. A skiving blade 220 that extends over the entire width of the collection/harvest element separates a very thin boundary layer from the main fluid stream; spacing 217 may be between 10 and 40 um. If a somewhat larger spacing 217 is required due to boundary layer issues, some degree of downstream back pressure in harvest output 214 may be applied in order to maintain the desired flow ratio between the two exit outputs 212 and 214 (e.g. about 100:1) but this must be done while maintaining the desired localized shear forces (discussed below). Channel dimensions and flow levels in collection zone 207 are optimized for target cell rolling (e.g. ˜2.5 dyne/cm²). The initial portion of the harvest zone 209 may still have a selectin coating so that target cells continue to roll, and its spacing 217 is designed to maintain approximately the same 2.5 dyne/cm² shear force. Zone 210 is not treated with selectin chemistry, and gradually increases in depth so that by the time flow is in zone 211 the local shear force level is significantly lower that the threshold where cell rolling behavior ceases (e.g. about 0.25 dyne/cm²). Additional forms of kinetic energy such as heat or vibration may be selectively applied in zones 210 and/or 211 to further encourage cell separation from attractant chemical 33 into the harvest output 214. Further, electrostatic potential may be considered to induce cell migration either toward or away from a particular surface.

For each transit of an ‘average bolus’ of fluid, 1 to 2% of fluid is extracted as harvest output 214, and this fluid has a modest proportion of the target cells, but with extremely high specificity. Over a large number (e.g. 10 or more) of transits a large majority of target cells will come close enough to the selectin-coated surface to be marginated and induced into rolling behavior and thus be captured and harvested. Depending on design parameters such as the length of collection zone 207 and use of secondary structures in the general channel to induce cell contact with attractant chemical 33, the surface area of the collection chamber 206 may be significantly smaller that the area required to collect a therapeutically effective number of stem cells in the case of a single pass system. This may in practice be less than 25 in² (161 cm²) and preferably on the order of 10 in² (65 cm²). As previously discussed, additional techniques such as pulsatile flow and use of additional surface treatments may further optimize performance of the collection/harvest element.

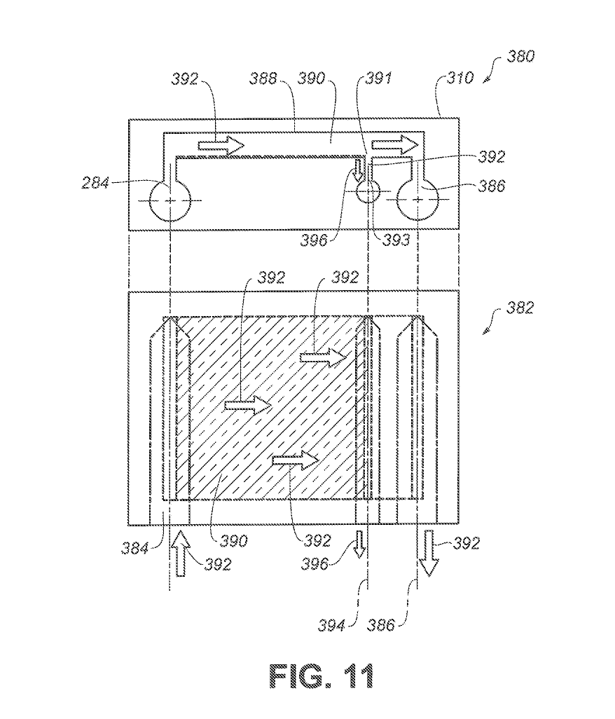

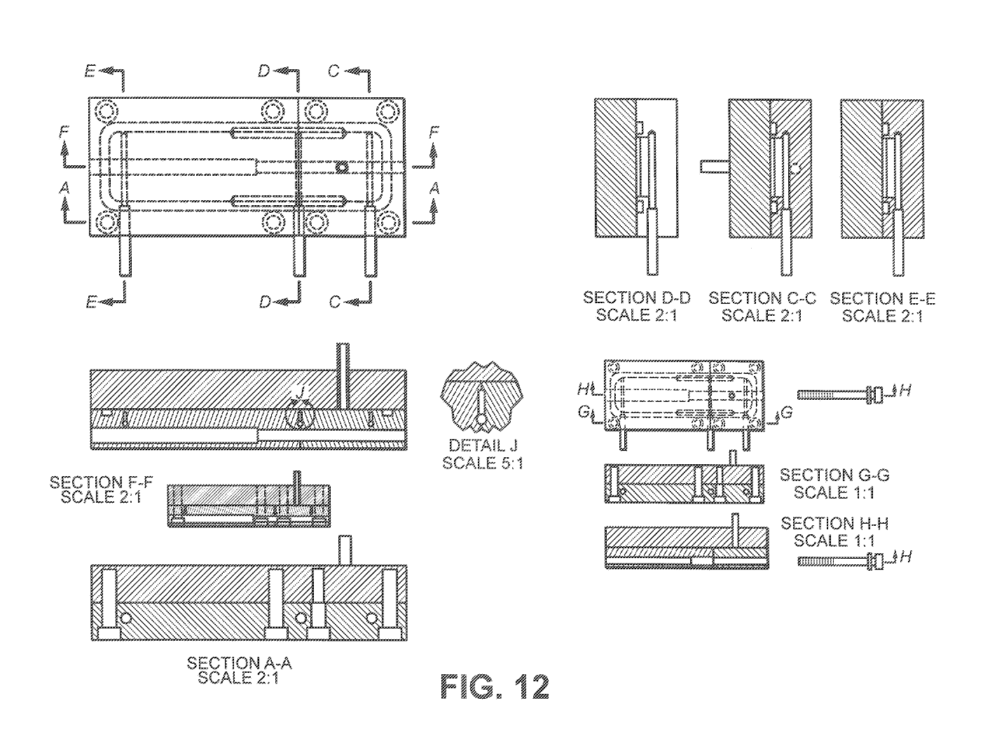

FIG. 10 is a block diagram of an overall cell collection system 300 that is based on active pressure sensing and pumping of both the primary circulating flow and the harvest flow in order to accurately control surface shear forces for optimal target cell collection. Referring to FIG. 10, a source 302 of cells as previously described communicates with pump 306 through line 304. Additionally if it is desired to have make-up fluid in the form of buffer fluid in order to maintain overall fluid volume this may be housed in container 320 that communicates via valve 322 with pump 306. Alone or in combination source 302 and/or buffer from container 320 are pumped through supply line 308 into collection module 310. As disclosed previously, up to 99% of this fluid travels via return line 312 back to the patient cell source 302 where it re-mixes and over time transits collection module 310 multiple times. As noted below, collection module 310 will be shown in FIG. 11 as having collection and harvest of cells on only one of its planar surfaces, but those skilled in the art will appreciate that both top and bottom surfaces may be coated, and dual harvest lines may be used; one for cells collected from the bottom surface of collection module 310 and one for cells collected from the top surface of collection module 310.

In one embodiment of this invention, the internal design of collection module 310 is such that two sets of conditions are inherently met; proper surface shear forces are maintained in flux-rolling areas and harvest areas of collection module 310, and the desired ratio of flow in harvest flow 314 to return line 312 (e.g. around 1%) is also maintained. Since this may not be easily accomplished, especially in consideration of changes in fluid viscosity, there is provided an alternative embodiment that includes a either a pressure or flow sensor, and/or a valve 316, to control the above parameters within desired ranges for optimal cell harvesting. Harvested cells are collected in container 318 for further processing and/or therapeutic use.

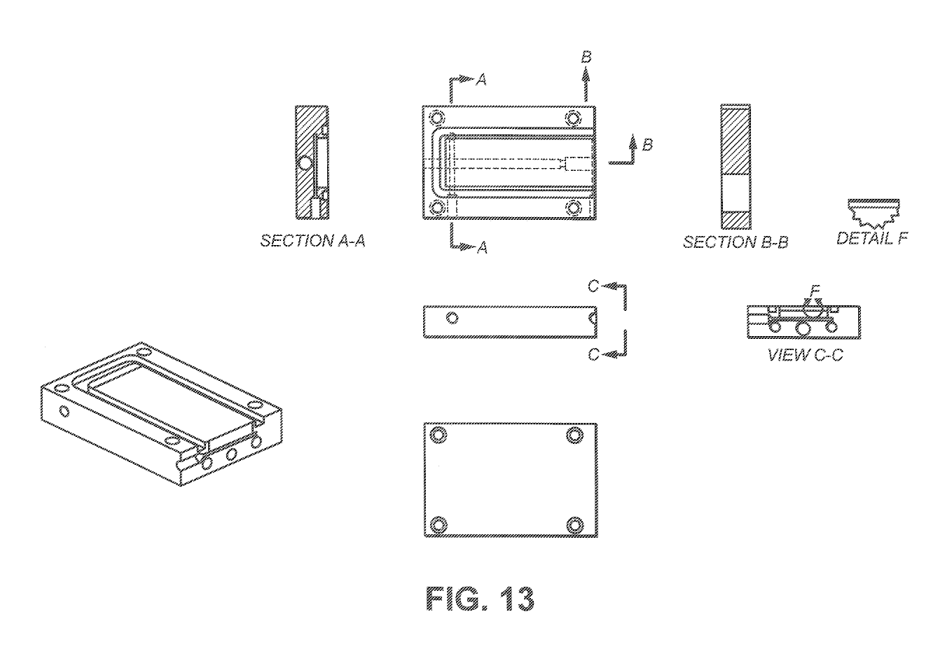

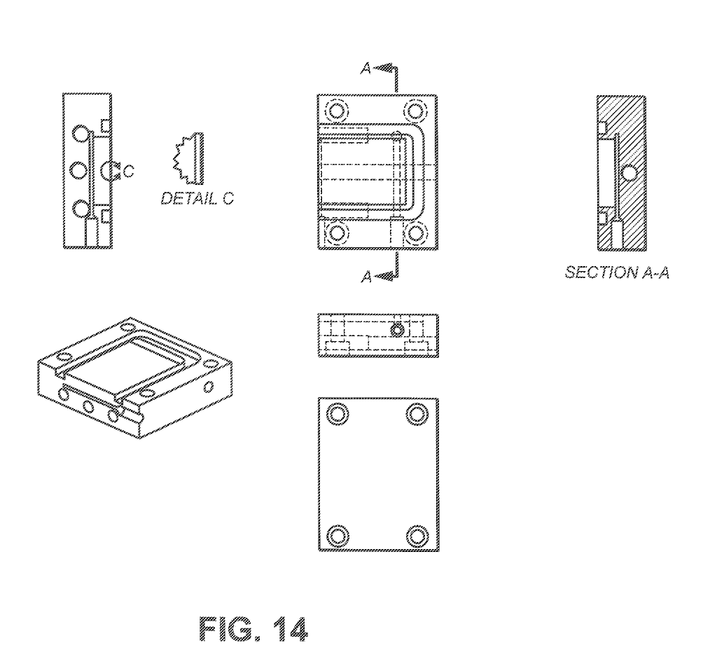

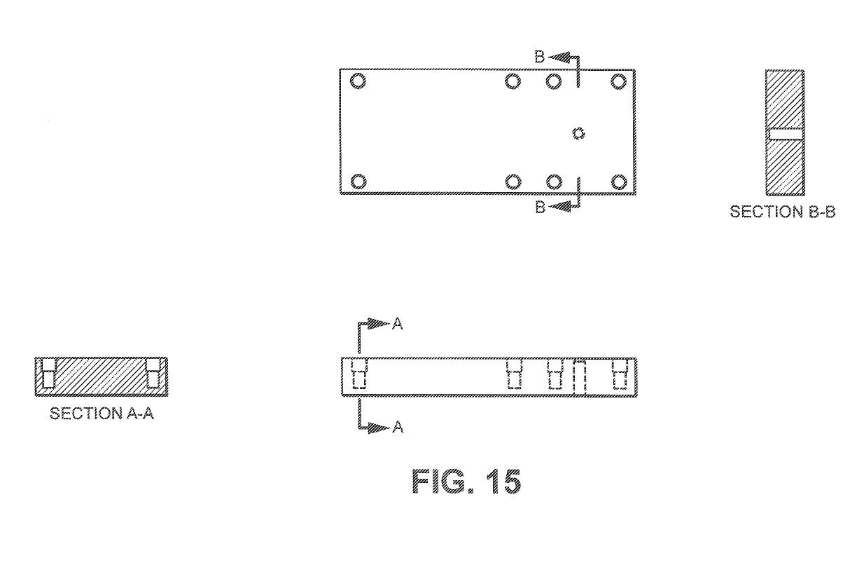

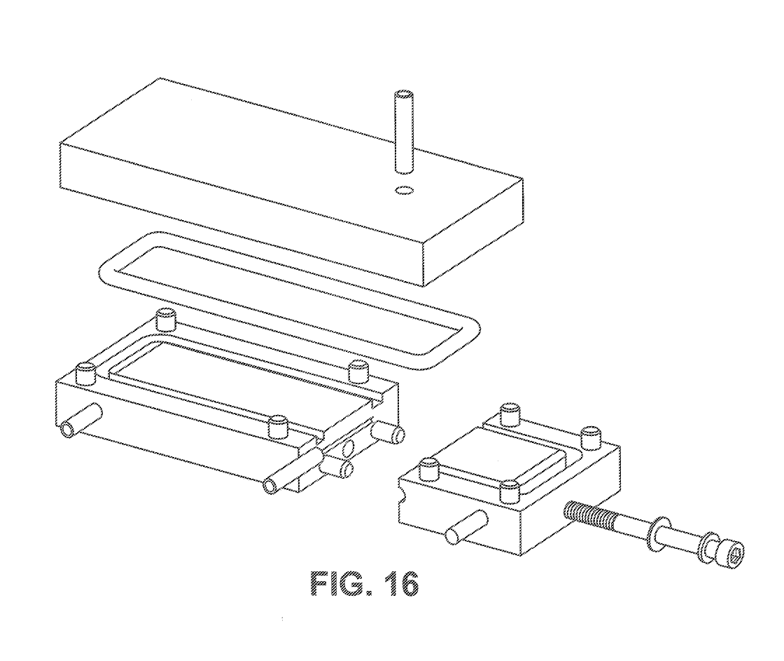

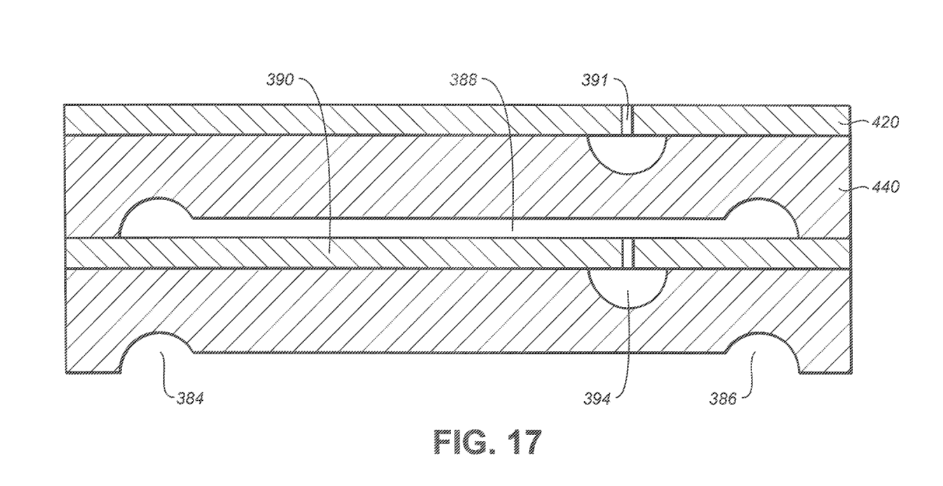

FIG. 11 is a schematic drawing of one embodiment of the planar cell collection module of this invention, showing flux rolling and collection of cells on one surface of the channel rather than both surfaces; those skilled in the art will understand it is simple to further develop the module to provide for two-surface collection thus increasing capacity by a factor of two. Side view 380 and top view 382 of cell collection module 310 share common notation with FIG. 10 by way of functional description, and are a schematic representation of the actual mechanical design shown in FIGS. 12-16.

Referring to FIG. 11, a planar cell collection channel 388 is fed in a recirculating manner by transverse input path 384 and transverse exit path 386; the recirculating flow being represented by arrows 392, and being operationally identical to recirculating output 212 from FIGS. 9A and 9B. Transverse harvest port 394, carrying harvest flow 396 is operationally identical to harvest output 214 in FIGS. 9A and 9B. Thus the functionality of the collection module shown in the embodiment of FIG. 11 (and in the CAD design shown in FIGS. 12-16) is topologically identical to that shown in FIGS. 9A and 9B, while being easier to manufacture in practice. Channel 388 is shown in side view 380 as having an attractant (e.g. selectin) on only one surface but, as discussed earlier, a secondary harvest port similar to port 394 could be placed on the opposing side of the channel to double the available surface area for flux rolling and cell collection.

Harvest flow 396 extends the full width of channel 388, and has an initial thickness on the order of 40 um, as opposed to the approximately 200 um thickness of the primary collection channel 388. In side view 380 harvest flow 396 is shown having an initial narrow section 391 followed by a tapering section 393. The dimensions of sections 391 and 392 may be adjusted to achieve proper flow balance between the primary recirculating flow 388 and the harvest flow 396 (e.g. between 100:1 and 40:1) without secondary controls since orifice flow is approximately a 4^{th} power function of hydraulic diameter, and since for a narrow planar channel the hydraulic diameter is closely related to the channel thickness. Thus if the ratio of channel thickness is 5:1 (based on 200 um vs. 40 um dimensions discussed above) the natural flow ratio will be approximately (0.2)⁴ or around 1.6%. This value may be further adjusted by small changes in design dimensions or by a secondary flow control means either in the primary recirculating flow 388 or in the harvest flow 396.

As discussed earlier, some portion of target cells will engage in flux rolling across the selectin-coated surface 390, due both to the biochemical attraction mechanism and the fact that surface shear force will be controlled at around 2.5 dyne/cm² in the main channel 388. As these target cells reach harvest port 394 they will follow the harvest flow 396, and as the dimension of the slit increases from narrow section 391 through tapering section 393, and into harvest port 394, the effective hydraulic diameter will increase significantly, in turn reducing the surface shear force to far below the level (e.g. 2.5 dyne/cm²) required to maintain flux rolling. Thus all target cells that have been marginated and induced into flux rolling on surface 390 will leave the surface and will be collected in container 318 shown in FIG. 10. Since the harvest flow will be on the order of 1% per recirculation cycle of the overall fluid volume, and due to the heightened population of target cells in the harvest flow 396, the amount of non-target cells collected will be well under 1% per cycle. This may be further reduced by other means (e.g. fluidic, acoustic, and/or electrostatic) that may be applied at or near the harvest slit, but should not be necessary in order to achieve an extremely high ratio of target to non-target cells in the harvest flow 396.

Additional means (not shown in the figures but disclosed in various cited references) may be used to enhance mixing by creating vortex flow in the main channel of the cell collection module. This may be achieved in a device having only one channel surface coated with adherent (e.g. selectin) molecules by creating ridges, herringbone structures, or other non-uniformities on the opposing channel surface. These non-uniformities disrupt the otherwise pure laminar flow and present added opportunities for target cells to come into close proximity with the adherent molecules, thus enhancing the portion of target cells that engage in flux rolling required to harvest them. In a collection module having both surfaces of the primary channel coated with adherent chemistry, one or more structures may be placed within the channel (e.g. at the center, about 100 um from each surface of a 200 um deep channel); these structures may be in the form of microscopic vanes or other perturbations that achieve the necessary vortex flow within the channel to enhance engagement of target cells in flux rolling on the two channel surfaces.

FIGS. 12 through 16 are included for reference purposes only, to indicate proper scale of an initial prototype of one embodiment and to demonstrate the practicality of manufacture either by machining or injection molding.

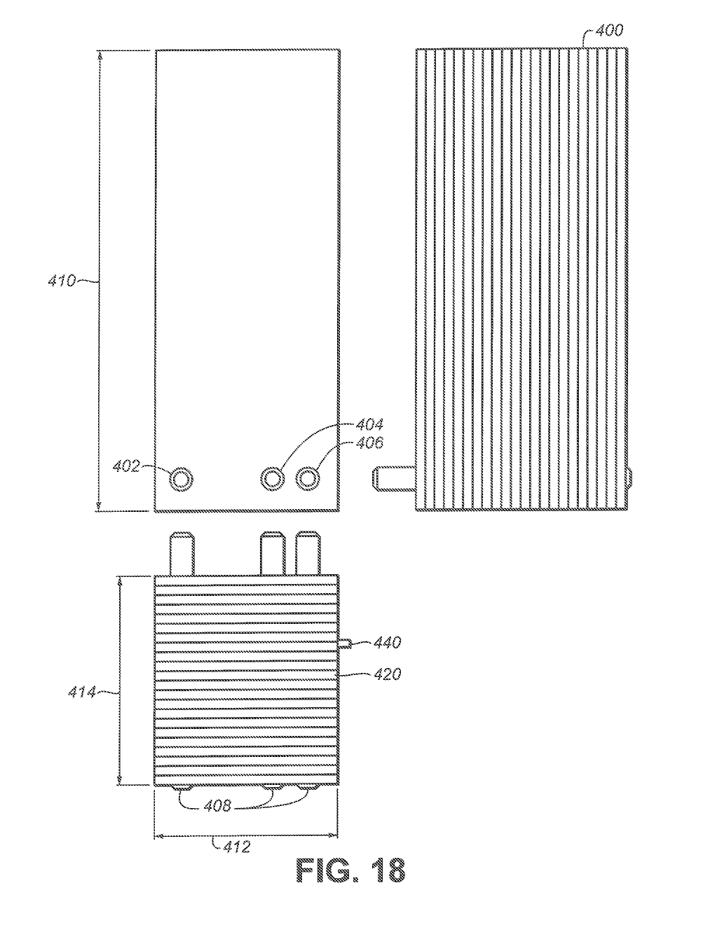

FIGS. 17 through 23 depict an embodiment that permits an increase in cell collection efficiency by a factor of up to 100. Referring again to FIG. 11, the dimension of the coated surface 390 that is parallel to flow 392 must be long enough to permit engagement of target cells in rolling behavior, but any additional length will not increase the performance of the system. The amount of flow at a fixed shear rate will be proportional to the product of the dimension of coated surface 390 that is perpendicular to flow 392, and the channel height. There are practical limits to increases in the dimension perpendicular to flow since the shape factor of the consumable collection will then become unmanageable if it is too wide and if the channel height becomes significantly larger than e.g. 0.200 mm the portion of target cells in the circulating flow that come close enough to the attractant-coated surface 390 to become marginated will be so low that a very large number of recirculation cycles will be required to capture the majority of these target cells. Thus it will be appreciated that optimization of the collection system (from a design standpoint) will be a matter of compromise between a) the lateral dimensions of the attractant-coated surface, b) channel height, c) flow velocity, d) the portion of flow that is shunted into the harvest slit during each recirculation cycle, and e) the number of recirculation cycles. Another way of stating this compromise is that if the above factors a) through e) are properly balanced, the cell collection system will (from a commercial standpoint) be able to f) collect a significant majority of target cells from the fluid volume, g) with a high degree of specificity (e.g. with a low proportion of non-target cells), h) in a reasonable timeframe, and using a disposable collection module that has reasonable i) size and j) cost. FIGS. 17 through 23 depict an embodiment that permits multiple collection surfaces to be stacked, having a single entry port, a single recirculation port, and a single harvest port. This stacked module may be configured to have multiples of 2 through as much as 100 or more in throughput compared to the prototype module disclosed in FIGS. 12 through 16.

Referring to FIGS. 11 and 17, like features will be described, each having the same operational function but having a different feature shape and/or dimension. FIG. 17 depicts two sets of channel element 440 and spacer element 420 that in combination carry out the functions shown in side view 380 of FIG. 11. More than two sets of these elements may be stacked in order to increase module performance. The narrow section 391 shown in both FIGS. 11 and 17 has an identical function, as do collection channels 388, attractant-coated surfaces 390, input path 384, harvest ports 394, and recirculation ports 386. It should be noted that in FIG. 17 spacer elements 420 and channel element 440 are shown having exaggerated dimensions in order to permit understanding of their functionality; in later figures these elements will be shown with proper dimensions that permit a far higher throughput in a small volume.

The depth of collection channel 388 shown in FIG. 17 is designed to maintain a surface shear force near 2.5 dyne/cm² as previously disclosed, and narrow section 391 is designed to permit about 1% of the overall flow to be diverted into it, while also decreasing the shear force beyond to it to well below 2.5 dyne/cm² (e.g. 0.25 dyne/cm² or lower). The ratio of flow exiting narrow section 391 may be inherent (e.g. a 4^{th} power function of hydraulic diameter) or may be controlled by way of a downstream valve or pump as shown in FIG. 10 and previously discussed. Individual sets of spacer element 420 in combination with channel element 440 may be designed to carry out the same functions as module 310 shown in FIG. 11 and described in detail in previous disclosure. If multiple sets of spacer element 420 and channel element 440 are stacked in such a way that multiple input paths 384 are fluidly connected, multiple recirculation channels 386 are fluidly connected, and multiple harvest ports 394 are also fluidly connected, the multiple sets of spacer elements 420 and channel elements 440 may be connected to a single set of input, recirculation, and harvest ports.

In the embodiment shown in FIGS. 17 through 23 the approach taken to fluidly connect multiple sets of spacer elements 420 and channel elements 440 is analogous to that taken in designing multi-layer electronic circuit boards, where ‘vias’ are used to connect one or more components in a vertical manner, orthogonal to the plane of individual circuit board layers.

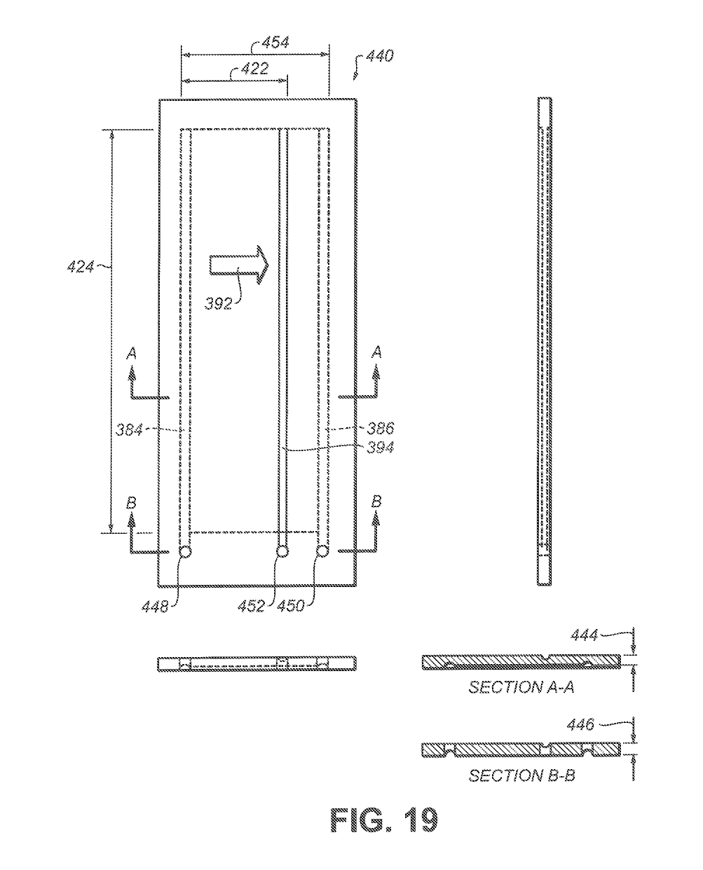

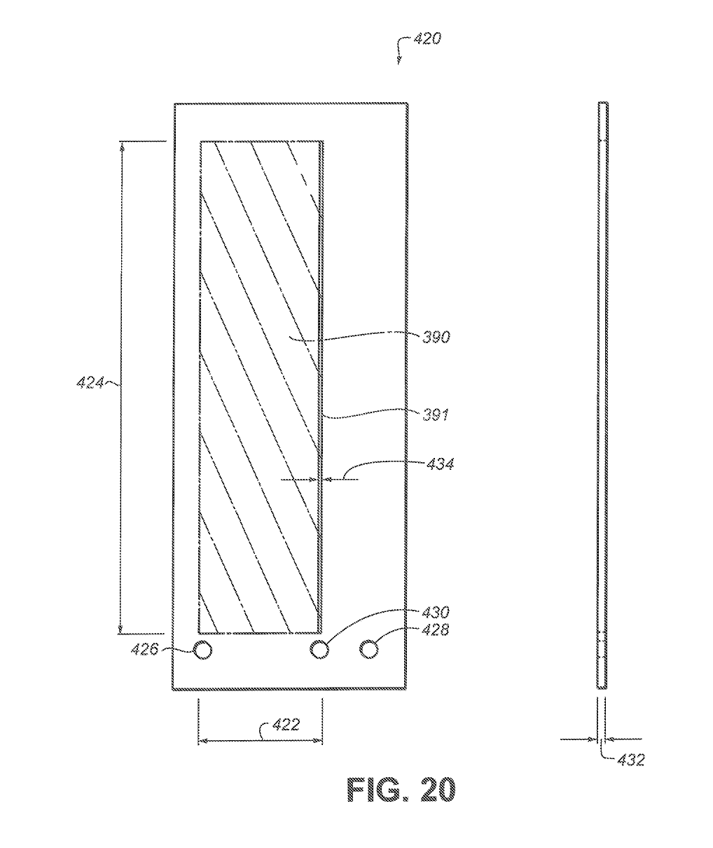

FIG. 18 depicts one embodiment of a high throughput cell collection device that may be connected to the same external pumping and supply components shown in FIG. 10. Cell collection assembly 400 comprises multiple sets of channel element 440 interleaved with spacer element 420; in this case there are 20 sets of these elements. The approximate dimensions of assembly 400 are as follows: length 410 may be approximately 120 mm, width 412 may be approximately 50 mm, and height 414 may be approximately 56 mm. Each of these dimensions may be adjusted to create a different form factor; height 414 may be changed by using more or less than 20 sets of spacer element 420 and channel 440; length 410 may be changed in order to adjust the lateral width of the flow path. Adjustments to dimensions of height 414 and/or length 410 will either increase or decrease the volumetric flow rate of fluid through the assembly thus directly influencing throughput of the assembly. Dimension of width 412 is related to the length of the flow path across the attractant-coated surface; this dimension must be sufficient to engage target cells in rolling but any greater length will not increase the per-pass efficiency of cell collection. However, path length (path length 422 shown in FIG. 19 and FIG. 20) may be different for each of the specific attractants used in the cell collection device so for example a path length 422 suitable for collection of stem cells from peripheral blood may differ from a path length that is suitable for collection of circulating tumor cells.

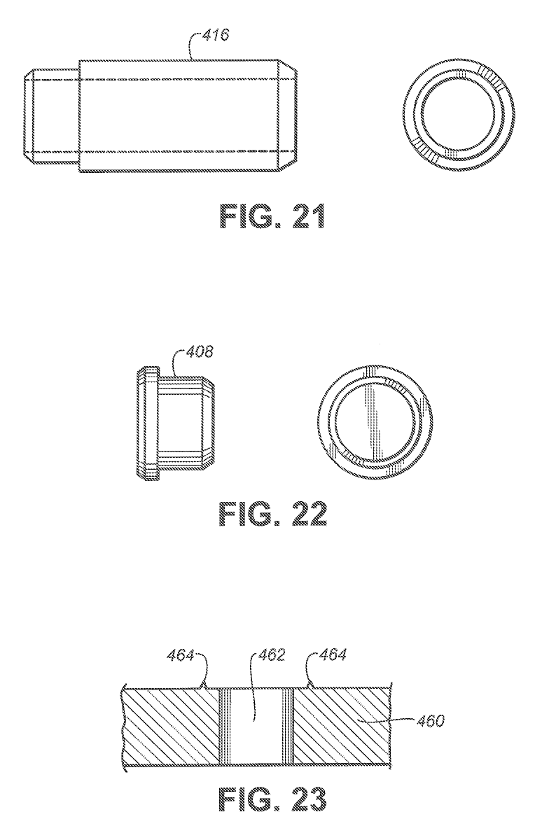

Also shown in FIG. 18 are input port 402, recirculation port 406, and harvest port 404. These ports connect the collection assembly 400 to the external pumping, fluid supply, and harvest components of the system, and are shown in more detail as component 416 in FIG. 21. Each of port 402, 404, and 406 fluidly connects with multiple sets of vias that are holes through each of channel elements 440 and spacer elements 420 that make up the collection assembly. In this manner, ports 402, 404, and 406, pressed into respective vias in the upper-most channel element 440, convert it into a top plate that seals the top of the assembly to prevent leakage, and provides fluid connection to the other components of the cell collection system. Further, the vias in the lower-most channel plate 440 are sealed with plugs 408, shown in this FIG. 18, and also shown in more detail in FIG. 22. These plugs convert the lower-most element 440 into a bottom plate that serves to seal the assembly and prevent leakage.

FIG. 19 depicts channel element 440 that has the same width and length dimensions as the collection assembly 400, and a thickness 446 of approximately 2.0 mm. Thickness 446 is chosen as a compromise between compactness, rigidity, and ease of fabrication and assembly. Thus its thickness will depend on the mode of attachment (e.g. adhesive and/or ultrasound) between it and spacer element 420, the method of fabrication (e.g. injection molding), and the polymer used in its manufacture (e.g. polycarbonate, acrylic, styrene).

For clarity, the direction of fluid flow in the collection channel is shown by arrow 392. As previously discussed, the length 410 of assembly 400 is influenced by path width 424, and the width 412 of assembly 400 is influenced by path length 422, both shown here in FIG. 19. Also shown in FIG. 19 are input path 384, exit path 386, and harvest port 394 that are fluidly connected to vias 448, 450, and 452, respectively. Section view B-B in FIG. 19 depicts the fluid connection between these channels and their respective vias. Section view A-A depicts the collection channel (wherein fluid flow is in the direction indicated by arrow 392) created by a recess shown by thickness 444, and having an area defined by path width 424 and dimension 454 which is the spacing between input path 384 and exit path 386. For illustrative purposes, dimension 454 may be about 35 mm and collection channel thickness 444 may be about 0.200 mm, and is optimized to maintain surface shear force to about 2.5 dyne/cm².

FIG. 20 depicts spacer element 420 that is manufactured from a thin sheet material such as plastic (e.g. polycarbonate, acrylic, styrene) or glass or metal, the choice of which depends on ease of attachment of the attractant chemistry, ease of fabricating a very thin narrow section 391, and ease of attachment to channel element 440 using adhesive and/or ultrasound methods. Thickness 432 of spacer element 420 may be on the order of 0.500 mm and its length and width may be the same as like dimensions for channel element 440. Narrow section 391 (also shown in FIG. 17) has a width 434 of approximately 0.040 mm, a path width 424 of approximately 100 mm, and upon assembly of the overall collection assembly 400 is directly aligned with harvest channel 394 shown in FIG. 19. Due to its small dimension and the criticality of maintaining uniformity, narrow section 391 may be fabricated using laser machining or other precise methods; the relatively small thickness 432 of spacer element 420 is intended to make this fabrication step as simple as possible. The top surface of spacer element 420 has a coated surface 390 of attractant chemistry shown as the cross-hatched area defined by path width 424 and path length 422. Input via port 426, recirculation via port 428, and harvest via port 430 are located so as to align with vias 448, 450, and 452 shown in FIG. 19, and with ports 402, 406, and 404 shown in FIG. 18, respectively.

In comparing the throughput of the prototype embodiment shown in FIGS. 12 through 16 with the multi-layer collection assembly shown in FIG. 18 and discussed above, the factors determining throughput include the width of individual flow paths and the number of paths. In the prototype, the path width was designed to be 20 mm; in the multi-layer assembly it is 100 mm, a factor of 5 wider. The multi-layer assembly shown in FIG. 18 comprises 20 sets of spacer elements 420 and channel elements 440, creating 20 flow paths working in parallel. Thus the overall functional throughput of the multi-layer assembly is 100 times higher than the throughput of the prototype module. This may of course be adjusted based on need by either increasing or decreasing path width 424, and by adjusting the number of pairs of spacer elements 420 and channel elements 440 used in the assembly.

FIG. 21 depicts a port component 416, also shown in FIG. 18, that is used in three places per assembly 400. It may be a turned metal part or a molded polymer part, and may be assembled into the top channel element 440 by press fitting, adhesive bonding, solvent bonding, ultrasonic welding, or a combination of these techniques.

FIG. 22 depicts plug 408, also shown in FIG. 18, that is also used in three places per assembly 400. It may be fabricated and affixed in like or different manner as is component 416, thus converting the lower-most channel element 440 into the bottom sealing plate of assembly 400.

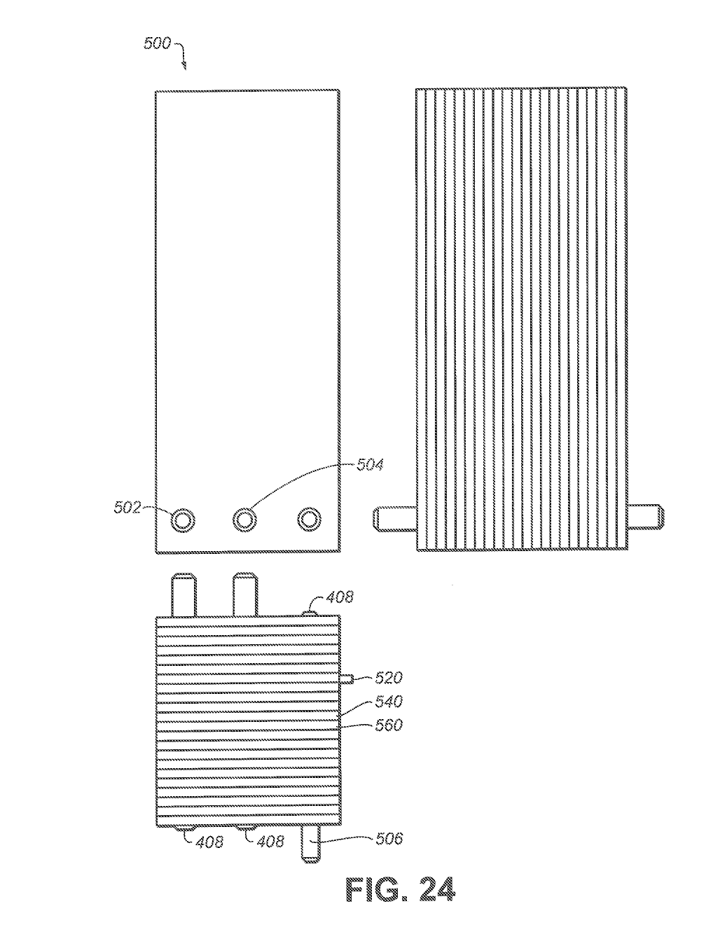

FIG. 23 shows a typical cross-section 460 of either a channel element 440 or a spacer element 420. Feature 462 is a void in channel element 440 that may be one of the several vias, or it may be the narrow section 391, or may alternatively be a feature that does not pass completely through cross-section 460, as would be the case with one of the several transverse channels such as input path 384, exit path 386, and harvest port 394 that have a semi-circular section as depicted in FIG. 19 but may also have a different cross-sectional shape. In the case where ultrasonic sealing is chosen as means for affixing the multiple spacer elements 420 and channel elements 440 together and for preventing leakage of fluid outside of the desire fluid path, ultrasonic concentrators may be used. As shown in FIG. 23, ultrasonic concentrator 464, positioned adjacent to feature 462, serves to concentrate the thermal energy used to seal cross-section 460 to another element above it (not shown). Those skilled in the art will understand that ultrasonic concentrators may be arrayed in a path surrounding vias, channels, and slits; in cooperation with an ultrasound horn having the appropriate shape. Under mechanical compression and upon application of ultrasonic energy the concentrators between multiple element sets will melt during the application of ultrasound energy and bond the components together permanently, and will seal them to control fluid flow. In FIG. 23 concentrators 464 are shown on the top surface of cross-section 460. Since it may be preferred that spacer element 420 be manufactured from sheet stock and since channel element 440 will likely be manufactured by injection molding, a preferred approach (not shown) may be to mold concentrators on both the top and bottom surfaces of channel element 440 so as to provide attachment and sealing to adjacent spacer elements 420 on both its upper and lower surface.

As an alternative, pressure-sensitive, thermally-activated, UV-activated, or solvent-bonding adhesives may be used to create the same mechanical bonding and fluid sealing as may be achieved by ultrasonic bonding. As a further alternative, one or more adhesive methods may be used in combination with ultrasonic sealing.

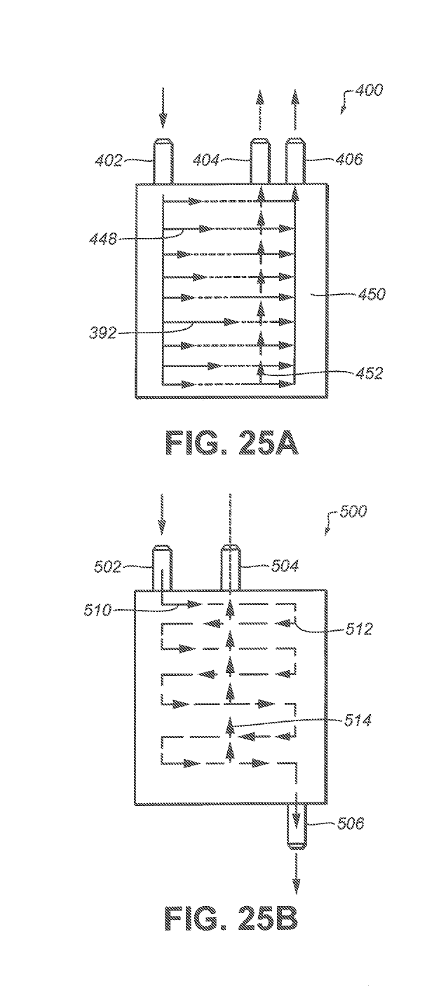

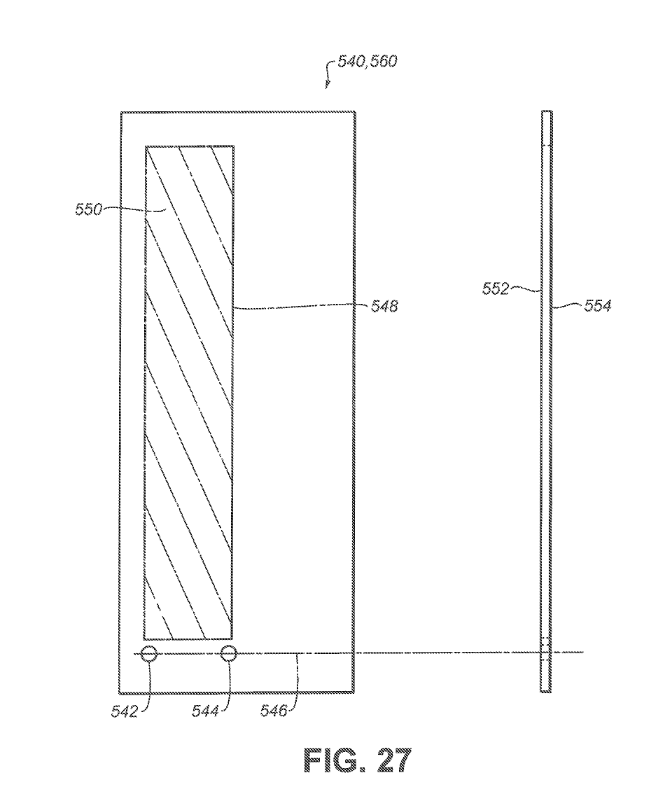

In yet another embodiment, depicted in FIG. 24, multi-layer cell collection assembly 500 is designed to operate in serial rather than parallel fashion, as does assembly 400, disclosed above. Assembly 400 is intended to be used in a manner that increases throughput by circulating fluid in parallel across e.g. 20 attractant-coated surfaces within the 20 channels 388 depicted in FIG. 17. Conversely, assembly 500 is designed increase throughput by circulating fluid in series across the e.g. 20 coated surfaces. In this manner, one operational cycle will take e.g. approximately 20 times longer, but will collect target cells at 20 points during the cycle since the fluid flow will cross narrow section 391 in each of the 20 collection channels 388. Components found in assembly 500 are in most respects identical to those in assembly 400 so only exceptions will be discussed herein. Referring to FIG. 24, it will be noted that the centerline position of harvest port 504 is midway between the centerline positions of input port 502 and recirculation/exit port 506. This is because the distance between ports 502 and 504, and the distance between ports 506 and 504 are designed to be equal to or slightly larger than the path length 422 shown in FIG. 19 which is the minimal distance required to engage cells in flux rolling on the attractant-coated surface within the module. Ports 502, 504, and 506 are made from port component 408 as shown in FIG. 22. Channel element 520 has the same function as channel element 440 in FIG. 19 but with the same symmetry in channel spacing as discussed above; this will be covered in more detail below and in FIG. 26. There are two different spacer elements 540 and 560 that are physically identical except for the surface upon which the attractant chemistry is coated; this will be covered in more detail below and in FIG. 27. Spacer element 540 is coated on its top surface as shown in FIG. 27 and assembled upright, while spacer element 560 is coated on its bottom surface and assembled inverted.

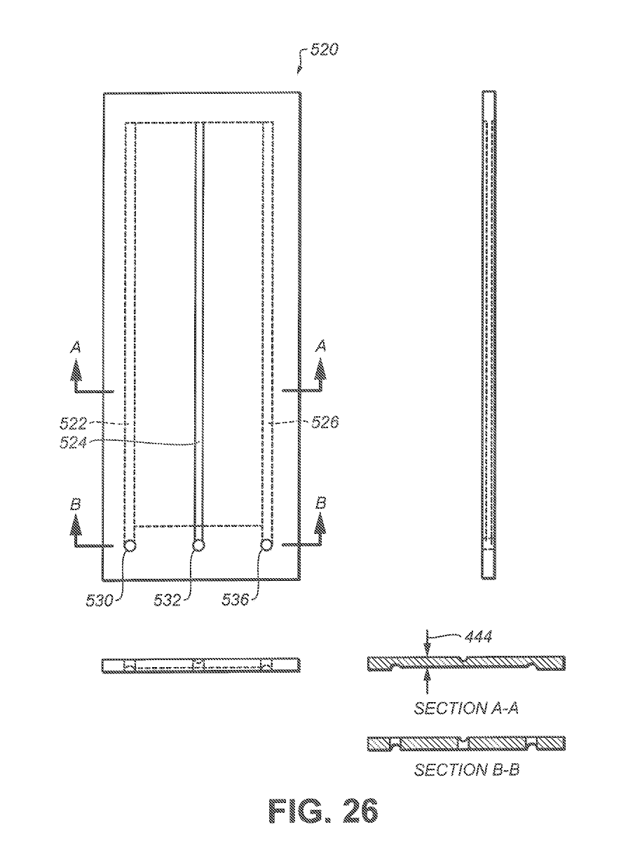

Referring to FIG. 25A and FIG. 25B, a comparison is made between the routing of fluid in collection assemblies 400 and 500, respectively. In assembly 400 fluid travels vertically down from input port 402 as shown by the line of vias 448, thence across the e.g. 20 collection channels in parallel as shown by arrow 392. A small portion (e.g. about 1%) of the fluid exits vertically through the line of vias 452 and out the harvest port 404, while the remainder exits the recirculation port 406 after moving vertically up the line of vias 450. As shown in FIG. 25B, fluid enters input port 502, through the first via, and thence laterally to the right as shown by arrow 510, crossing an attractant-coated surface, passing a harvest slit where a portion of the fluid carrying target cells exits through a via, flowing vertically as shown by arrow 514 and thence out the harvest port 504. The remainder of the fluid continues to flow laterally until it reaches the right-hand end of the collection channel, moves down through a via, and then reverses direction as shown by arrow 512. At this level the attractant-coated surface is on the right side of the axis of harvest port 504 and in this manner target cells rolling on the surface are again harvested in vias and out harvest port 504. It should be noted that FIG. 25A and FIG. 25B are for reasons of clarity shown with fewer than the e.g. 20 sets of channel and harvest elements that comprise assemblies 400 or 500.

FIG. 26 depicts channel element 520 used to construct collection assembly 500. In most respects it is identical in design to channel element 440 shown in FIG. 19, except that vias 530, 532, and 536 are equally spaced, as are channels 522, 524, and 526. As in channel element 440, the recess with thickness 444 shown in Section A-A that creates the collection channel extends fully between input channel 522 and return/exit channel 526.

FIG. 27 depicts spacer elements 540 and 560 that are used in alternating fashion to create collection assembly 500. Via 544 aligns with via 532 in channel element 520. As shown in FIG. 27 via 542 in spacer element 540 aligns with via 530 in channel element 520, and if inverted, via 542 in spacer element 560 will align with via 536 in channel element 520. Position 546 shows where a third via would be positioned but by intent there is no third via. Harvest slit 548 is identical in dimension and construction as is narrow section 391 in spacer element 420 but it is at the centerline of spacer element 420, creating the shaded area 550 indicating the extent of the attractant-chemistry coating. Note that spacer element 540 is coated on its top surface 552 and spacer element 560 is coated on its bottom surface 554.

Collection assembly 500 is created by separating channel elements 520 with alternating spacer elements 540 and 560. In this manner, the missing via (shown at position 546 in FIG. 27) blocks fluid movement in an upward direction, thus causing the downward movement and reversal of flow direction at each level in the module. In this manner, module 500 operates in serial fashion rather than parallel. All other features, dimensions, and methods of manufacture for collection assembly 500 are the same as previously disclosed for assembly 400.

Due to the 4^{th} power dependence of flow on hydraulic diameter, uniform flow across the width of the collection channel in each of the embodiments shown in FIGS. 11 through 27 depends on very close control of channel thickness. For example, a variation of as little as 40 microns across a channel that is 200 microns thick will result in a flow ratio of about 2.2. This will in turn mean that some parts of the collection area coated with attractant chemical will either be well above or well below the 2.5 dyne/cm² surface shear force that is so critical to effective operation of the system.

Referring once again to FIG. 23 it will be appreciated that ultrasonic concentrators 464, when compressed and heated by ultrasonic energy, will melt along with a small area of the mating surface. This will cause a very slight separation between the two surfaces after bonding and this separation will contribute to a lack of flatness that can affect the thickness of the collection channel as discussed immediately above. Those skilled in the art will appreciate that if there is a small recess (not shown in FIG. 23) adjacent to concentrators 464, either on one or both sides, and that if the cross section of the recess is equal to or larger than the cross section of the concentrator, excess polymer will flow into the recess and permit the components to be attached fixedly without distortion.

And referring once again to Section A-A in FIGS. 19 and 26, it will be understood that recess with thickness 444 that forms the collection channel, must be held to a high degree of uniformity, as discussed above. While not shown in FIG. 19 or 26 it will be appreciated by those skilled in the art that small features in the form of dimples or other shapes that extend from the top of the recess to the bottom of the channel element will serve to maintain channel thickness to a very high degree of precision.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The following references are hereby incorporated by reference in their entirety:
Nanostructured Surfaces to Target and Kill Circulating Tumor Cells While Repelling Leukocytes; Mitchell et al; 2012 Journal of Nanomaterials, Volume 2012 (2012),
Isolation of Circulating Tumor Cells Using a Microvortex-Generating Herringbone-Chip; Stott et al; 18392-18397/PNAS/Oct. 26, 2010/vol. 107/no. 43
Optimal Design of Microfluidic Networks Using Biologically Inspired Principles; Barber, Emerson; Microfluid Nanofluid (2008) 4:179-191 DOI 10.1007/s10404-007-0163-9
Microfluidic Shear Devices for Quantitative Analysis of Cell Adhesion; Lu et al; Anal. Chem. 2004, 76, 5257-5264
Controlled Viable Release of Selectively Captured Label-Free Cells in Microchannels; Gurkan et al; Royal Society of Chemistry 2011; DOI: 10.1039/c11c20487 d
Microfluidic System for Studying the Interaction of Nanoparticles and Microparticles with Cells; Farokhzad et al; Anal. Chem. 1005, 77, 5453-5459
Microfluidic Models of Vascular Functions; Wong et al; Annu. Rev. Biomed. Eng. 2012.14:205-230
On Laminar Flow in Microfabricated Channels with Partial Semi-Circular Profiles; Federspie, Valenti; Open Journal of Applied Sciences; 2012, 2, 28-34
Mathematical Model for the Effects of Adhesion and Mechanics on Cell Migration Speed; DiMilla et al; Biophysical Journal; 1991, Vol. 60, 15-37

1. A cell collection assembly comprising:
a cell collection channel that is split at a junction into a bypass port for passing non-flux rolling cells to a receptacle and a harvesting port for harvesting flux rolling cells to a container, the cell collection channel comprising a collection zone upstream from the junction, the collection zone coated with an attractant chemical, the collection zone having a first width selected to produce a shear force that causes flux rolling of target cells;
the harvesting port comprising a harvest zone disposed adjacent the collection zone and downstream from the junction and a release zone disposed adjacent to, and downstream from, the harvest zone, the release zone having a second width selected to produce a shear force that stops flux rolling of the target cells.

2. The cell collection assembly of claim 1 wherein the cell collection channel is downstream from a first pumping zone and the receptacle is a second pumping zone, the first pumping zone and second pumping zone being fluidly connected by the cell collection channel.

3. The cell collection assembly of claim 2, wherein the first pumping zone and second pumping zone comprise a flexible material.

4. The cell collection assembly of claim 1 wherein the attractant chemical is a selectin.

5. The cell collection assembly of claim 1 wherein the bypass port has a width that passes a first volume of fluid and the harvesting port has a width that passes less than 10% of the first volume of fluid.

6. The cell collection assembly of claim 1 wherein the shear force that stops flux rolling is less than about 0.5 dyne per square centimeter.

7. The cell collection assembly of claim 1 wherein the cell collection assembly comprises two or more sets of the cell collection channels, each of which is fluidly connected in parallel.

8. The cell collection assembly of claim 1 wherein the cell collection assembly comprises two or more sets of the cell collection channels, each of which is fluidly connected in series.

9. The cell collection assembly of claim 2 wherein the first pumping zone and second pumping zone each comprise a first, a second and a third flexible laminate layer that provides a first fluid-containing area and a second fluid-containing area, the second fluid-containing area being selectively pressurizable to apply pressure to a fluid within the first fluid-containing area.

10. A method for enriching a fluid sample comprising non-stem cells and stem cells, the method comprising steps of:
pumping a fluid sample from a source to a collection assembly, the collection assembly comprising an intake port that receives the fluid sample from the source, a return line that returns a first portion of the fluid sample from the collection assembly to the source and a harvest port that sends a second portion of the fluid sample from the collection assembly to a container;
permitting stem cells and non-stem cells to pass through a collection zone in the collection assembly wherein:
the stem cells engage in flux rolling on a surface coated with an attractant chemical such that the flux rolling causes the stem cells to pass through the harvest port;
the non-stem cells do not engage in flux rolling on the surface and pass through the return line;
releasing the stem cells from the surface after the stem cells have passed through the harvest port;
passing the stem cells into the container after the stem cells have been released from the surface to produce a harvested sample that is enriched in the stem cells relative to the fluid sample;
recirculating the first portion of the fluid sample that passed through the return line to the source.

11. The method of claim 10 wherein the harvest port comprises a narrow section with a first width and a tapering section with a second width, the first width being selected to produce a first shear force to cause flux rolling, the second width being selected to produce a second shear force to stop flux rolling, the tapering section being disposed downstream from the narrow section.

12. The method of claim 11, wherein the second shear force to stop flux rolling produced by the second width is less than the first shear force.

13. The method of claim 12, wherein the first shear force to cause flux rolling produced by the first width is about 2.5 dynes per square centimeter.

14. The method of claim 12 wherein a volume of the second portion of the fluid sample is less than about 10% of the first portion of the fluid sample.

15. The method of claim 12 wherein a volume of the second portion of the fluid sample is less than about 2% of the first portion of the fluid sample.

16. A method for enriching a fluid sample comprising non-target cells and target cells, the method comprising steps of:
pumping a fluid sample from a first pumping zone, through a collection zone and into a second pumping zone, the collection zone comprising a surface coated with an attractant chemical that causes a first portion of the target cells to engaged in flux rolling on the surface;
pumping the fluid sample from the second pumping zone, through the collection zone and into the first pumping zone, thereby causing a second portion of the target cells to engaged in flux rolling on the surface.

17. The method as recited in claim 16, further comprising a step of releasing the target cells from the collection zone by eluting the collection zone with an elution fluid.
